(19) United States
(12) Patent Application Publication
(10) Pub. No.: US 2026/0191991 A1
(43) Pub. Date: Jul. 9, 2026

(54) COMPSTATIN ANALOGS FOR VECTOR-BASED THERAPY

(71) Applicants: Despina YANCOPOULOU, Attica (GR); AMYNDAS PHARMACEUTICALS US LLC, Jackson, WY (US)

(72) Inventor: Despina YANCOPOULOU, Attica (GR)

(21) Appl. No.: 19/132,449

(22) PCT Filed: Nov. 22, 2023
(86) PCT No.: PCT/IB2023/061764

§ 371 Date: May 23, 2025

Related U.S. Application Data
(60) Provisional application No. 63/427,882, filed on Nov. 24, 2022.

(51) Int. Cl. A61K 48/00; A61K 9/00; A61K 38/00; A61P 27/02; C07K 7/64; C07K 14/71; C07K 16/22; C12N 15/86

(52) U.S. Cl. CPC A61K 48/005; A61K 9/0043; A61K 48/0075; A61K 48/0083; A61P 27/02; C07K 7/64; C07K 14/71; C07K 16/22; C12N 15/86; A61K 38/00; C07K 2317/522; C07K 2317/55; C07K 2317/76; C07K 2319/00; C12N 2750/14143

(57) ABSTRACT

Compounds comprising peptides capable of binding C3 protein and inhibiting complement activation are disclosed. The compounds comprise compstatin analogs that can be vectorized for vector-mediated therapy, for example, viral-vector mediated gene therapy, either as stand-alone treatment for diseases involving aberrant complement activation, or in combination with other viral vector mediated gene therapies to reduce vector-induced complement activation and to confer added or synergistic therapeutic benefit when combined with other drug modalities in the same or multiple vectors. Nucleic acids encoding the compstatin analogs are also disclosed, as are vectors, pharmaceutical compositions and methods of using the compstatin analogs.

### FIELD OF THE INVENTION

This invention relates to activation of the complement cascade in the body. In particular, this invention provides compstatin analogs that can be vectorized for vector-mediated therapy, for example, viral-vector mediated gene therapy, either as stand-alone treatment for diseases involving aberrant complement activation, or in combination with other viral vector mediated gene therapies to reduce vector-induced complement activation and to confer added or synergistic therapeutic benefit when combined with other drug modalities in the same or multiple vectors. These novel compstatin natural amino acid-containing molecules can be used for therapeutic C3 modulation in systemic or local applications.

### BACKGROUND OF THE INVENTION

Various publications, including patents, published applications, technical articles and scholarly articles are cited throughout the specification. Each of these cited publications is incorporated by reference herein, in its entirety.

Viral vector gene therapies use modified viruses as drug-delivery vehicles to introduce specific DNA sequences into cells. The technology has long drawn interest for its potential advantages over traditional modalities. Many types of therapeutic agents (for example, therapeutic peptides, enzymes, antibodies, and regulatory RNAs) can be encoded in DNA sequences that can be rapidly designed and synthesized once a target is identified. These vectored therapies to be delivered once or much less frequent than traditional therapeutic modalities, thereby also increasing patient compliance.

Nearly all gene therapies currently available use one of three vector types: adeno-associated-virus (AAV) vectors, adenovirus vectors, or lentivirus (retrovirus) vectors. AAV and adenovirus vectors are typically used in gene therapies that are directly administered to patients by infusion or local administration (in vivo), with AAV being the most popular vector for areas outside of oncology and vaccines. While the applicability of viral vector-based gene therapy is expanding into new disease areas and clinical conditions, there is an unmet clinical need for developing effective countermeasures that can mitigate vector-induced immune-related adverse events.

For instance, a maladaptive host immune response to the AAV capsid or transgene, involving both innate and adaptive immune pathways, can limit the transduction efficacy and therapeutic efficacy of AAV-based therapies. Complement activation has been implicated as a key pathogenic driver of acute immunotoxicities associated with AAV gene therapy (Hamilton B A et al, 2021, Front Immunol. 2021 May 17; 12:675897. doi: 10.3389/fimmu.2021.675897. eCollection 2021.PMID: 34084173; Muhuri M et al, J Clin Invest, 2021 Jan. 4; 131 (1): e143780. doi: 10.1172/JCI143780.PMID: 33393506; Smith C J et al, Front Immunol. 2022 Sep. 16; 13:999021. doi: 10.3389/fimmu.2022.999021. eCollection 2022.PMID: 36189251). Pre-existing neutralizing or non-neutralizing IgM and IgG antibodies against the AAV capsid or transgene can potently trigger complement activation via the classical pathway, leading to C3 activation, amplification of complement responses via the alternative pathway and downstream activation of the terminal pathway (i.e., direct MAC-mediated cytotoxicity). C3 activation takes center stage in this cascade of events that collectively limit the transduction efficiency of AAV particles, compromising the clinical efficacy of AAV therapy. C3 activation can lead to macrophage activation, increased AAV uptake via CR3-dependent phagocytic processes and direct AAV toxicity via downstream lytic pathway activation. Also, C3-derived fragments can modulate antigen presentation pathways and B cell responses that affect anti-AAV antibody generation and promote AAV-directed cytotoxic T cell responses. In view of the multifaceted role of C3 in these processes that impact on both AAV viability and effective tissue homing, C3 inhibition appears to be a central therapeutic modality to enhance the clinical potential of systemic AAV-based therapies.

The human complement system contributes to various pathologies, from autoimmune, age-related, and inflammatory disorders to transplant- and biomaterial-induced complications, making it a prime target for therapeutic intervention. Despite growing interest, the development of complement-targeted drugs has been slow, with two related anti-C5 antibodies (eculizumab, ravulizumab) approved for the treatment of paroxysmal nocturnal hemoglobinuria (PNH) and other indications long remaining the only clinical options. It was only in 2021, with the FDA and EMA approval of pegcetacoplan (Empaveli®/Aspaveli®, Apellis), when a second class of complement inhibitors with distinct mechanism became available. Compared to existing therapies, pegcetacoplan acts upstream in the complement cascade by impairing the activation of the central component C3 to provide a broader control of complement effectors. An extension of therapeutic intervention points within the cascade has been highly anticipated in view of the diverse involvement of complement in pathology. Recently, two more complement inhibitors, the anti-C1s mAb (sutimlimab, Enjaymo) and a small-molecule C5aR1 antagonist (Avacopan, Tavneos) have been approved for complement mediated diseases such as cold agglutinin disease and ANCA-associated vasculitis, respectively.

Complement primarily acts as a rapid host defense system that eliminates microbial intruders and apoptotic cells. After initiation by various means, including immune complexes (classical pathway) or microbial signatures (lectin pathway), the cascade converges at the activation of the plasma protein C3 by convertases. C3 cleavage releases the anaphylatoxin C3a and produces an opsonic fragment (C3b), which covalently attaches to the target cell surface. Concerted binding of the proteases factor B (FB) and factor D (FD) to C3b generates the main C3 convertase (i.e., C3bBb) to activate more C3. In absence of regulators, this process feeds an amplification loop (alternative pathway) that rapidly opsonizes surfaces with C3b. While C3b and its degradation fragments are directly involved in phagocytic and adaptive immune signaling, C3b also provides a platform for the formation of C5 convertases. Cleavage of C5 produces the inflammatory mediator C5a and generates membrane attack complexes (MAC) that lyse or damage susceptible cells. Whereas these potent effector functions provide an important layer of antimicrobial defense, any excessive or misguided complement activation may drive clinical complications by inducing tissue damage, inflammation and adverse immune reactions. Depending on the disorder, a pathway- or effector-specific inhibition may prove sufficient, while other conditions require an approach that suppresses complement activity more broadly.

The compstatin family of C3 inhibitors is particularly suited for broad complement inhibition as it largely impairs convertase-mediated C3 activation by all pathways and prevents most effector generation. Compstatin was originally derived from phage display as a disulfide-bridged, 13-amino-acid peptide with micromolar binding affinity for C3 that was optimized for improved affinity, efficacy, and pharmacokinetic properties. Substituting residues in the cyclic core resulted in compstatin analogs with profoundly enhanced target affinity. Compstatin Cp05 (SEQ ID NO: 2) builds the base for pegcetacoplan (Empaveli®/Aspaveli®, Apellis), in which two Cp05 units are bridged by a 40-kDa PEG moiety to reduce renal elimination. Finally, backbone N-methylation and the addition of D-Tyr to the N-terminus yielded analog Cp40 (SEQ ID NO: 3), which featured picomolar affinity and an improved pharmacokinetic profile in absence of PEGylation.

Recently, new analogs of Cp40 have been developed, which possess enhanced solubility and improved pharmacokinetic profiles. PEGylation with small PEG moieties, or the addition of Lys residues, were demonstrated to increase the solubility of Cp40 at physiological pH (~7.4), without affecting the favorable C3 inhibitory activity of Cp40. In addition, the new Cp40-based compstatin derivatives showed similar or prolonged half-lives after subcutaneous (sc) administration into NHPs, resulting in longer saturation of plasma C3 (period of time in which the molar concentration of a Cp40 analog is equal to or above the molar concentration of plasma C3) as compared with Cp40. These improved properties facilitate sc administration of the Cp40-based derivatives, thereby leveraging patient compliance during chronic C3-targeted intervention, and also expand the possible routes for Cp40 delivery, thus widening its potential use in various indications. Recent studies in patients with severe COVID-19 have shown that Cp40 administration can confer complete and sustained systemic C3 inhibition, saturating the plasma concentration of C3 for the duration of treatment. C3 inhibition by Cp40 is followed by a broad anti-inflammatory effect and a pronounced attenuation of thrombogenic responses (i.e., Neutrophil Extracellular Trap (NET) release) (Skendros P, Germanidis G, et al, Sci Adv, 2022 Aug. 19; 8 (33): eabo2341. doi: 10.1126/sciadv.abo2341. Epub 2022 Aug. 17.PMID: 35977025). Moreover local (intragingival) administration of Cp40 to adult patients with periodontal inflammation resulted in significant reduction of key clinical indices of gingival inflammation and markers of inflammatory tissue injury, with the sustained therapeutic effect extending to 90 days after the discontinuation of treatment (Hasturk H et al, J Clin Invest, 2021 Dec. 1; 131 (23): e152973. doi: 10.1172/JCI152973.PMID: 34618684).

Though compstatin itself is a peptide composed of natural amino acids, the above-mentioned compstatin analogs contain unnatural constituents, the inclusion of which has improved their potency many hundred-fold as compared with compstatin, and has imparted numerous pharmacokinetic benefits. These advantages, together with their small size, makes Cp40 and its derivatives particularly attractive for pharmaceutical development. However, the presence of the unnatural constituents in these analogs precludes their expression and manufacturing from a DNA sequence in a viral vector or other expression vectors. As such, it would be an advance in the art to develop new analogs that are amenable to vectorization, yet have comparable activity and pharmacokinetic profiles as the highly effective Cp40 and derivatives.

### SUMMARY OF THE INVENTION

The present invention provides analogs of the complement-inhibiting peptide, compstatin that are amenable to vectorization as single therapeutic modalities or as fusion moieties in combination with other therapeutic modalities (such as anti-VEGF agents) and manufacturing by expression, as well as possessing robust complement inhibitory activity and desirable pharmacokinetic properties.

One aspect of the invention features a compstatin analog comprising a peptide having an amino acid sequence Xaa1-[Cys-Ile-Trp-Gln-Asp-Trp-Gly-Xaa2-His-Arg-Cys]-Xaa3-Xaa4 (SEQ ID NO:4), wherein Xaa1 is absent or comprises the dipeptide Tyr-Ile, Xaa2 is Ala or Glu, Xaa3 is absent or is Ile, and Xaa4 is absent or represents one, two or three Lys residues, and wherein the Cys residues form a disulfide bond, to form a cyclic peptide comprising the sequence within the brackets. In particular, the compstatin analog can have an amino acid sequence Tyr-Ile-[Cys-Ile-Trp-Gln-Asp-Trp-Gly-Xaa2-His-Arg-Cys]-Ile-Xaa4 (SEQ ID NO:5), wherein Xaa2 is Ala or Glu and Xaa4 represents two or three Lys residues. Embodiments include SEQ ID NO:6 and/or SEQ ID NO: 7, as well as any one of SEQ ID NOS: 8-16.

An aspect of the invention features a polynucleotide comprising a sequence that encodes the compstatin analogs or peptides described above. In one embodiment, the polynucleotide encodes SEQ ID NO:6 or SEQ ID NO:7. In one embodiment, the polynucleotide is selected from SEQ ID NO: 17 and SEQ ID NO:18.

In certain embodiments the compstatin analog polynucleotide is disposed within an expression cassette or a vector. The vector can be an expression vector. It can be adapted for expression in a procaryotic or eucaryotic expression system. In certain embodiments, the vector is used in gene therapy and is selected from retrovirus, adenovirus, adeno-associated virus (AAV), and herpes simplex virus-1. In certain embodiments, the vector is an adeno-associated viral (AAV) vector. The AAV vector can be of type AAV1, AAV2, AAV3, AAV4, AAV5, AAV6, AAV7, AAV8, AAV9, AAV10, or AAV11 vector, or any variant thereof.

Another aspect of the invention features a vector, or a multiplicity of vectors, at least one of which includes a polynucleotide that encodes a compstatin analog comprising SEQ ID NO:4, SEQ ID NO:5, SEQ ID NO:6, SEQ ID NO:7, or any one of SEQ ID Nos 8-16. The compstatin analog polynucleotide can have a sequence of SEQ ID NO:17 or SEQ ID NO:18. The vector can also include at least one insertion site for at least one transgene for delivery of gene therapy. The vector can be selected from retrovirus, adenovirus, adeno-associated virus (AAV), and herpes simplex virus-1. In certain embodiments, the vector is an adeno-associated viral (AAV) vector. The AAV vector can be selected from an AAV1, AAV2, AAV3, AAV4, AAV5, AAV6, AAV7, AAV8, AAV9, AAV10, or AAV11 vector, or any variant or combination thereof.

In certain embodiments, the above-described vector comprises at least one other transgene for gene therapy. The transgene can encode, for example, a therapeutic protein, enzyme, hormone, blood coagulation factor, cytokine, or growth factor. In various embodiments, the gene therapy is for the treatment of a blood disorder, an eye disorder, autoimmune disease, a muscle disorder, a neurological disorder, or cancer. In certain embodiments, the compstatin analog polynucleotide and/or transgene are adapted for tissue or organ-specific expression.

In certain embodiments, the compstatin analog polynucleotide and the transgene are arranged on the vector to produce a fusion protein comprising the compstatin analog and the transgene product. The fusion protein can comprise the compstatin analog directly linked to the transgene product, or it can comprise the compstatin analog linked to the transgene product via a linker or spacer.

In particular embodiments, the transgene encodes a VEGF inhibitor. The VEGF inhibitor may comprise an extracellular domain of a VEGF receptor. In other embodiments, the VEGF inhibitor comprises an antibody fragment selected from the group consisting of a Fab, F(ab′)2, Fv, scFv, or a single domain antibody. The antibody fragment can be a Fab comprising a VL domain, a CL domain, a VH domain, and a CH1 domain. In certain embodiments, the vector of encodes a fusion protein comprising the compstatin analog fused directly or indirectly to one, two, three, or four of: (i) the VL of the Fab; (ii) the CL of the Fab; (iii) the VH of the Fab; or (iv) the CHI of the Fab. The vector can encode a fusion protein comprising the compstatin analog fused directly or indirectly to the N-terminus of the VL of the Fab, the C-terminus of the CL of the Fab, the N-terminus of the VH of the Fab, or the C-terminus of CHI of the Fab.

Another aspect of the invention features a pharmaceutical composition comprising at least one vector as described above, and a pharmaceutically acceptable carrier. The pharmaceutical composition can be formulated for administration by a route selected from subcutaneous, intradermal, intravenous, intraocular (including intravitreal and subretinal), intracerebral, intraperitoneal, intramuscular injection, periodontal administration (including gingival administration or intrapapillary infiltration injection), intranasal, epidural, oral, sublingual, intrathecal, intravaginal, transdermal, rectal, by inhalation, or topical.

The pharmaceutical composition can be formulated for systemic administration, or it can be formulated for local administration. The local administration can be to the brain and/or central nervous system, eye, lungs and/or respiratory system, heart and/or vascular system, lymphatic system, kidney, spleen, pancreas, liver, gastrointestinal system, periodontal tissue, skin, bone, joint or synovial fluid, or any combination thereof. In addition to the aforementioned vectors, the pharmaceutical composition can contain a compstatin analog peptide as described herein, which may have a sequence selected from SEQ ID NO:4, SEQ ID NO:5, SEQ ID NO:6 and SEQ ID NO:7.

Another embodiment features a pharmaceutical composition comprising a compstatin analog having a sequence selected from SEQ ID NO:4, SEQ ID NO:5, SEQ ID NO:6 and SEQ ID NO: 7 in a pharmaceutically acceptable carrier. This composition is useful for certain therapeutic applications as described herein. This pharmaceutical composition can be formulated for administration by a route selected from subcutaneous, intradermal, intravenous, intraocular (including intravitreal, subretinal), intracerebral, intraperitoneal, intramuscular injection, periodontal administration (including gingival administration or intrapapillary infiltration injection), intranasal, epidural, oral, sublingual, intrathecal, intravaginal, transdermal, rectal, by inhalation, or topical. It can be formulated for systemic or local administration, the local administration can be to the brain and/or central nervous system, eye, lungs and/or respiratory system, heart and/or vascular system, lymphatic system, kidney, spleen, pancreas, liver, gastrointestinal system, periodontal tissue, skin, bone, joint or synovial fluid, or any combination thereof. The above-described pharmaceutical composition containing a compstatin analog can further comprise at least one vector as described herein.

Another aspect of the invention features a kit comprising a plurality of pharmaceutical compositions, wherein at least one of the pharmaceutical compositions is a peptide-containing composition comprising a compstatin analog having a sequence selected from SEQ ID NO:4, SEQ ID NO:5, SEQ ID NO:6 and SEQ ID NO:7 in a pharmaceutically acceptable carrier, and at least one of the pharmaceutical compositions is a vector-containing composition comprising a vector for gene therapy as described herein, in a pharmaceutically acceptable carrier. In one embodiment, at least one of the peptide-containing composition and the vector-containing composition is formulated for systemic administration. In another embodiment, at least one of the peptide-containing composition and the vector-containing composition is formulated for local administration. In another embodiment, the peptide-containing composition is formulated for a selected route of administration and the vector-containing composition is formulated for a route of administration that is different than the route of administration selected for the peptide-containing composition. The kit may also include instructions for administering the peptide-containing composition on a schedule that is different from the schedule for administering the vector-containing composition.

Another aspect of the invention, the kit includes a plurality of pharmaceutical compositions, each of which is a vector-containing composition comprising a vector as described herein, in a pharmaceutically acceptable carrier. These kits can include pharmaceutical compositions formulated for systemic administration, local administration, or both. These kits can include pharmaceutical compositions formulated for different routes of administration. They can also include instructions for administering one composition on a schedule that is different from the schedule for administering another composition.

Another aspect of the invention features a method of treating a subject having or at risk of a complement-mediated disorder, the method comprising administering to the subject a composition that includes at least one vector comprising a compstatin analog polynucleotide that encodes a compstatin analog of SEQ ID NO:4, SEQ ID NO:5, SEQ ID NO:6 or SEQ ID NO:7. The subject can be a non-human primate or a human. In certain embodiments, after the administration of the composition, a level of complement activity in the subject or in a biological sample from the subject is reduced relative to a level before the administration of the composition, or in an equivalent subject who has not been administered the composition. The level of complement activity can be reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration or in the equivalent subject who has not been administered the composition.

In certain embodiments, the composition is administered systemically to the subject. In other embodiments, the composition is administered locally to tissues or organs of the subject.

In certain embodiments, the vector is an adeno-associated viral (AAV) vector. The AAV vector can be an AAV1, AAV2, AAV3 (e.g., AAV3B), AAV4, AAV5, AAV6, AAV7, AAV8, AAV9, AAV10, or AAV11 vector.

In certain embodiments, the complement-mediated disorder is a chronic disorder. In certain embodiments, the complement-mediated disorder involves complement-mediated damage to red blood cells, optionally wherein the disorder is paroxysmal nocturnal hemoglobinuria or atypical hemolytic uremic syndrome. In certain embodiments, the complement-mediated disorder is an autoimmune disease, optionally wherein the disorder is multiple sclerosis. In certain embodiments, the complement-mediated disorder involves the kidney. These disorders can include membranoproliferative glomerulonephritis, lupus nephritis, IgA nephropathy (IgAN), primary membranous nephropathy (primary MN), C3 glomerulopathy (C3G), or acute kidney injury. In certain embodiments, the complement-mediated disorder involves the central or peripheral nervous system or neuromuscular junction. Examples include neuromyelitis optica, Guillain-Barre syndrome, amyotrophic lateral sclerosis, multifocal motor neuropathy, or myasthenia gravis. In certain embodiments, the complement-mediated disorder involves the respiratory system, The respiratory disorder can be characterized by pulmonary fibrosis. In certain embodiments, the complement-mediated disorder involves the vascular system. The vascular system disorder can be characterized by vasculitis.

In certain embodiments of the method, the composition is administered to the eye of a subject suffering from an eye disorder. The composition can be administered intravitreally. The eye disorder can be age-related macular degeneration (AMD). In certain embodiments, eye has one or more of (i) geographic atrophy, (ii) wet AMD (riii) geographic atrophy and wet AMD, or (iv) intermediate AMD.

The above described method can include the steps of: (1) providing the subject; (2) administering the composition to the subject, whereby the compstatin analog is produced in the subject; and (3) measuring one or more parameters of the complement-mediated disorder. The measuring can be performed before, during and/or after administration of the composition. It can also, or alternatively, be performed on an equivalent subject who is not administered the composition.

In certain embodiments, the complement-mediated disorder is selected from the group consisting of atypical hemolytic uremic syndrome (aHUS); dense deposit disease (DDD); C3 glomerulonephritis (C3GN); C3 glomerulopathies; complement-mediated nephropathies and glomerular inflammatory diseases; age-related macular degeneration (AMD); eye disorder characterized by macular degeneration, choroidal neovascularization (CNV), retinal Neovascularization (RNV), proliferative vitreoretinopathy, glaucoma, uveitis, ocular inflammation, or any combination of these; paroxysmal nocturnal hemoglobinuria (PNH); cold agglutinin disease (CAD); warm antibody autoimmune hemolytic anemias (wAIHAs); sickle cell disease; transplant-associated thrombotic microangiopathies; rheumatoid arthritis (RA); systemic lupus erythematosus (SLE); autoimmune and autoinflammatory kidney diseases; autoimmune myocarditis; multiple sclerosis; traumatic brain and spinal cord injury; cerebral, intestinal and renal ischemia-reperfusion (IR) injury; spontaneous and recurrent pregnancy loss; anti-phospholipid syndrome (APS); Parkinson's disease; Alzheimer's disease; neurodegenerative inflammatory conditions underpinned by aberrant synaptic remodeling, microglial activity and cognitive decline; asthma; anti-nuclear cytoplasmic antigen-associated pauci-immune vasculitis (Wegener's syndrome); non-lupus autoimmune skin diseases such as pemphigus, bullous pemphigoid, and epidermolysis bullosa; post-traumatic shock; cancer; periodontitis; gingivitis; and atherosclerosis.

In certain embodiments, the method comprises administering more than one dose of the composition to the subject. The method can include administering doses of the composition to the subject at pre-determined time intervals. Such time intervals can be within hours, days, weeks or months of one another.

Another aspect of the invention features a method of treating a subject having or at risk of a disease or disorder having a complement-mediated component and one or more other components. The method comprises administering to the subject a composition that includes at least one vector comprising: (a) a compstatin analog polynucleotide that encodes a compstatin analog of SEQ ID NO:4, SEQ ID NO:5, SEQ ID NO:6 or SEQ ID NO:7 and (b) at least one transgene that encodes a gene product that treats the one or more other components. The subject can be a non-human primate or a human.

In certain embodiments, after the administration of the composition, a level of complement activation and/or the other component in the subject or in a biological sample from the subject is/are reduced or ameliorated relative to a level before the administration of the composition, or in an equivalent subject who has not been administered the composition. The level of complement activation and/or the other component can be reduced or ameliorated by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration or in the equivalent subject who has not been administered the composition.

In one embodiment, the other component is associated with a neovascular phenotype. In one embodiment, the composition is administered to the eye of a subject suffering from an eye disorder. In one embodiment, the eye disorder is age-related macular degeneration (AMD). In one embodiment for treatment of the eye disorder, the transgene encodes a VEGF inhibitor.

In certain embodiments, the method involves use of an adeno-associated viral (AAV) vector. The AAV vector can be an AAV1, AAV2, AAV3 (e.g., AAV3B), AAV4, AAV5, AAV6, AAV7, AAV8, AAV9, AAV10, or AAV11 vector. In certain embodiments, the transgene encodes a therapeutic protein, enzyme, hormone, blood coagulation factor, cytokine, or growth factor.

In certain embodiments, the compstatin analog polynucleotide and the transgene are arranged on a single vector to produce a fusion protein comprising the compstatin analog and the transgene product. The compstatin analog can be linked to the transgene product directly or via a linker or spacer. The fusion protein can include a VEGF inhibitor. In other embodiments, the compstatin analog polypeptide and the at least one other transgene are disposed on two or more separate vectors.

In certain embodiments, the vector comprising the compstatin analog and the vector or vectors comprising the at least one other transgene are administered by different routes. In certain embodiments, the vector comprising the compstatin analog is administered systemically and the vector or vectors comprising the at least one other transgene are administered locally. In certain embodiments, the vector comprising the compstatin analog is administered before, during or after administration of the vector or vectors comprising the at least one other transgene.

In certain embodiments, the method includes the steps of: (1) providing the subject; (2) administering the composition(s) to the subject, whereby the compstatin analog and the transgene are produced in the subject; and (3) measuring one or more parameters of the complement-mediated disorder or other component. The measuring can performed before, during and/or after administration of the composition(s), and/or on an equivalent subject who is not administered the composition. In certain embodiments of this method, the compstatin analog polynucleotide or the vector comprising the compstatin analog polynucleotide is replaced with the compstatin analog peptide itself.

Another aspect of the invention features a method of improving efficacy of gene therapy in a subject who will receive, is receiving or has received gene therapy. The method includes administering to the subject a vector comprising a compstatin analog polynucleotide that produces in the subject a compstatin analog having SEQ ID NO:4, SEQ ID NO:5, SEQ ID NO:6 or SEQ ID NO:7, thereby improving the efficacy of the gene therapy. In certain embodiments, the efficacy of the gene therapy is improved in the subject over a selected time period. The selected time period can be least about 1 week, 2 weeks, 4 weeks, 2 months, 3 months, 6 months, or one year, for example. In certain embodiments, the efficacy of the gene therapy is assessed by observing or measuring a decrease in an immune response against the gene therapy. In certain embodiments, the efficacy is assessed by observing or measuring improvement in transduction of a viral vector carrying a transgene. In certain embodiments, the efficacy is assessed by observing or measuring a decrease in complement-mediated clearance of a viral vector carrying a transgene. In certain embodiments, the efficacy of the gene therapy is measured by (i) comparing a subject receiving the vector comprising the compstatin analog polynucleotide with a control subject not receiving the vector comprising the compstatin analog polynucleotide, and/or (ii) comparing a subject during or after receiving the vector comprising the compstatin analog polynucleotide with the same subject before receiving the vector comprising the compstatin analog polynucleotide.

In certain embodiments of this method, the vector comprising the compstatin analog polynucleotide comprises at least one insertion site for at least one transgene for delivery of gene therapy. The vector can be selected from retrovirus, adenovirus, adeno-associated virus (AAV), and herpes simplex virus-1. In one embodiment, the vector comprising the compstatin analog polynucleotide is an adeno-associated viral (AAV) vector. The AAV vector can be an AAV1, AAV2, AAV3, AAV4, AAV5, AAV6, AAV7, AAV8, AAV9, AAV10, or AAV11 vector, or any variant or combination thereof.

In certain embodiments, the vector comprising the compstatin analog polynucleotide comprises at least one other transgene for gene therapy. In certain embodiments, the vector comprising the compstatin analog polynucleotide is different from another vector comprising another transgene for gene therapy, and the vectors are administered together. In other embodiments the vector comprising the compstatin analog polynucleotide is different from another vector comprising the other transgene for gene therapy, and the vectors are administered separately. Another embodiment of this aspect of the invention comprises replacing the compstatin analog polynucleotide or the vector comprising the compstatin analog polynucleotide with the compstatin analog peptide itself.

Other features and advantages of the present invention will be understood by reference to the detailed description that follows.

### BRIEF DESCRIPTION OF THE DRAWING

FIG. 1. Graph showing individual Grade 4 lesion ratios (expressed as % of week 2 Grade 4 lesions) following laser induced choroidal neovascularization (CNV) over time, in a macaque primate model. The arrow indicates intravitreal treatment (Day 15). Three eyes (K-2434, K-2437 and K-2485) were treated with compstatin analog CP50 and three eyes (K-2089, K-2482 and K-2468 were treated with vehicle.

### DETAILED DESCRIPTION OF THE ILLUSTRATIVE EMBODIMENTS

### Definitions

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used herein: Ac, acetyl group; BSA, bovine serum albumin; DCM, dichloromethane; DMF, dimethylformamide; ELISA, enzyme-linked immunosorbent assay; ESI, electrospray ionization; Fmoc, 9-fluorenylmethoxycarbonyl; MALDI-TOF-MS, matrix-assisted laser desorption ionization-time-of-flight mass spectrometry; NHP, non-human primate; PBS, Phosphate Buffered Saline; RP-HPCL, reversed-phase high-performance liquid chromatography; Sar, N-methyl glycine; s.c., subcutaneous; SPR, surface plasmon resonance; TFA, triflouroacetic acid; UPLC-ESI-MS, ultra-performance liquid chromatography-electrospray ionization-tandem mass spectrometry; VBS, Veronal buffered saline; WFI, water for injection.

The singular form of a word includes the plural, and vice versa, unless the context clearly dictates otherwise. The, the references “a”, “an”, and “the” are generally inclusive of the plurals of the respective terms. For example, references to “a compound” or “a method” includes a plurality of such “compounds” or “methods.” Similarly, the words “comprise”, “comprises”, and “comprising” are to be interpreted inclusively rather than exclusively. Likewise, the terms “include”, “including”, and “or” should all be construed to be inclusive, unless such a construction is clearly prohibited from the context.

The terms “comprising” or “including” are intended to include embodiments encompassed by the terms “consisting essentially of” and “consisting of.” Similarly, the term “consisting essentially of” is intended to include embodiments encompassed by the term “consisting of.” Moreover, the term “consisting essentially of” limits the scope of an embodiment to the specified components or steps and those components or steps that do not materially affect the basic and novel characteristics of the embodiment.

The term “about” as used herein when referring to a measurable value such as an amount, a temporal duration, and the like, is meant to encompass variations of ±20% or ±10%, in some embodiments ±5%, in some embodiments ±1%, and in some embodiments ±0.1% from the specified value, as such variations are appropriate to make and use the disclosed compounds and compositions.

The term “compstatin” as used herein refers to a peptide comprising SEQ ID NO:1, I[CVVQDWGHHRC]T (cyclic C2-C12 by way of a disulfide bond indicated by the brackets). The term “compstatin analog” refers to a modified compstatin comprising substitutions of amino acids amenable to expression from DNA, as described in greater detail herein. These analogs are sometimes referred to herein as being “proteinogenic.” When referring to the location of particular amino acids or analogs within compstatin or compstatin analogs, those locations are sometimes referred to as “positions” within the peptide, with the positions numbered from 1 (Ile in compstatin) to 13 (Thr in compstatin). For example, the Gly residue occupies “position 8.”

The terms “pharmaceutically active” and “biologically active” refer to the ability of the compstatin analogs of the invention to bind C3 or fragments thereof and inhibit complement activation. This biological activity may be measured by one or more of several art-recognized assays.

The term “gene therapy” as used herein encompasses therapies in which a disease or pathological condition is treated by introduction of exogenous DNA into cells of the individual with the disease or condition. Gene therapy includes, for example, (1) replacing a dysfunctioning or non-functioning gene with a healthy copy of the gene, (2) inactivating a disease-causing gene that is not functioning properly, leading to disease; and/or (3) introducing a heterologous nucleic acid into cells, tissues or organs in the body to produce a therapeutic agent to help treat a disease.

The terms “nucleic acid” and “polynucleotide” are used interchangeably herein to refer to all forms of nucleic acid, oligonucleotides, including DNA and RNA. Nucleic acids include genomic DNA, cDNA, antisense DNA/RNA, plasmid DNA, linear DNA, (poly- and oligo-nucleotide), chromosomal DNA, spliced or unspliced mRNA, IRNA, tRNA inhibitory DNA or RNA (RNAi, e.g., small or short hairpin (sh) RNA, microRNA (miRNA), small or short interfering (si) RNA, trans-splicing RNA, or antisense RNA), locked nucleic acid analogue (LNA), oligonucleotide DNA (ODN) single and double stranded, immunostimulating sequence (IS S), riboswitches and ribozymes. Nucleic acids include naturally occurring, synthetic, and intentionally modified or altered polynucleotides.

The term “transgene” refers to a nucleic acid that is intended or has been introduced into a cell or organism. Transgenes include any nucleic acid, such as nucleic acids encoding the compstatin analogs of the present invention “compstatin analog transgenes”, as well as any other heterologous nucleic acid encoding a protein, peptide or a nucleic acid (e.g., miRNA, etc.). The term transgene and heterologous nucleic acid/polynucleotide sequences are used interchangeably herein.

As used herein, the term “vector” refers to a nucleic acid molecule capable of transporting another nucleic acid to which it has been linked. One type of vector is a plasmid, which refers to a circular double stranded DNA loop into which additional DNA segments may be ligated. Another type of vector is a viral vector, wherein additional DNA segments may be ligated into the viral genome. Certain vectors are capable of directing the expression of genes to which they are operatively linked. Such vectors are referred to herein as “expression vectors.” One of ordinary skill in the art understands that a “viral vector”, as described herein, includes viral components in addition to a transgene.

An “expression control element” refers to nucleic acid sequence(s) that influence expression of an operably linked nucleic acid. Vector sequences and non-viral vectors can include one or more “expression control elements.” Typically, such elements are included to facilitate proper heterologous polynucleotide transcription and as appropriate translation (e.g., a promoter, enhancer, splicing signal for introns, maintenance of the correct reading frame of the gene to permit in-frame translation of mRNA and, stop codons etc.).

As used herein, “operably linked” refers to a relationship between two or more nucleic acid sequences where certain nucleic acid sequences (e.g., control elements) influence characteristics of another nucleotide sequence (e.g., influencing expression of a transgene). Operably linked sequences include both expression control elements that are included in or are contiguous with the transgene, and expression control elements that act in trans or at a distance to control expression of the transgene. Expression control elements as used herein include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. For example, as used herein, a nucleic acid sequence (e.g., a compstatin coding sequence or other transgene) and regulatory sequences are considered to be operably linked when they are covalently linked in such a way as to place the expression or transcription of the nucleic acid sequence under the influence or control of the regulatory sequences.

As used herein, “pharmaceutically acceptable salts” or “pharmaceutically acceptable esters” refer to derivatives of the disclosed compounds wherein the parent compound is modified by making an ester or an acid or base salt form, which is compatible with any other ingredients of the pharmaceutical composition, and which is not deleterious to the subject to which the composition is to be administered. Examples of pharmaceutically-acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically-acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds of the present invention may exist as zwitterions. All forms of the compounds, including free acid, free base, and zwitterions, are contemplated to be within the scope of the present invention.

As used herein, the term “local administration” or “local delivery”, in reference to delivery of a compstatin analog peptide or its encoding polynucleotide as described herein, refers to delivery that can rely upon transport of the peptide or polynucleotide to its intended target tissue or site via the vascular system.

As used herein, “intraocular administration” or “ocular administration” of a pharmaceutical composition includes any route of administration characterized by introduction into the eye, including subretinal administration and intravitreal administration. The term “intravitreal administration” of a pharmaceutical composition includes any route of administration characterized by introduction into the vitreous cavity of the eye. The “vitreous” is a gel-like substance within the vitreous cavity that fills the space between the lens and the retina and helps the eye maintain its shape.

As used herein, “intramuscular administration” of a pharmaceutical composition includes any route of administration characterized by introduction into the muscles.

As used herein, “periodontal administration” of a pharmaceutical composition refers to the administration within the tissues surrounding and/or around a tooth or teeth (e.g., by injection, topical application, or biodegradable implant), and includes “gingival administration” and “intrapapillary infiltration.” As used herein, “gingival administration” of a pharmaceutical composition refers includes any route of administration characterized by introduction to or into the gingiva, or gums. “Intrapapillary infiltration” or “intrapapillary infiltration injection” is a type of gingival administration that refers to administration of a pharmaceutical composition into the interdental papilla, which is the gingiva (gum) tissue that exists coronal to the free gingival margin on the buccal and lingual surfaces of the teeth.

As used herein, “oral administration” or “enteral administration” of a pharmaceutical composition includes any route of administration characterized by introduction into the gastrointestinal tract. “Oral administration” includes feeding by mouth as well as orogastric or intragastric gavage. “Oral administration” or “enteral administration” also may include sublingual, buccal, intranasal, pulmonary or rectal administration, among other routes known in the art.

The term “treating” refers to any indicia of success in the treatment or amelioration of the disease or condition. Treating can include, for example, reducing or alleviating the severity of one or more symptoms of the disease or condition, or it can include reducing the frequency with which symptoms of a disease, defect, disorder, or adverse condition, and the like, are experienced by an individual, such as a human patient.

The term “preventing” refers to the prevention of the disease or condition in an individual, such as a human patient. For example, if an individual at risk of developing an inflammatory disease is treated with the compounds and/or using the methods of the present invention and does not later develop the disease or condition, then the disease has been prevented in that individual.

The term “treat or prevent” is sometimes used herein to refer to a method that results in some level of treatment or amelioration of the disease or condition, and contemplates a range of results directed to that end, including but not restricted to prevention of the condition entirely.

The term “parameter” as used herein to refer to measuring any bodily function that is observable or measurable using suitable measuring techniques available in the art. As one having ordinary skill in the art will appreciate, measuring one or more “parameters” of bodily function can be used to detect a particular dysfunction as compared to the average normal parameters and can also be used to determine whether that bodily function has improved following or during treatment. Such parameters can be general, e.g., body temperature, blood pressure, pulse (heart rate), and breathing rate (respiratory rate), or they can be specific to a particular organ, tissue or disease or condition, e.g., functional test results from blood or other organs/tissues.

An “effective amount” or “sufficient amount” refers to an amount that provides, in single or multiple doses, alone or in combination, with one or more other compositions (therapeutic or immunosuppressive agents such as a drug), treatments, protocols, or therapeutic agents, a detectable response of any duration of time (long or short term), an expected or desired outcome in or a benefit to a subject of any measurable or detectable degree or for any duration of time (e.g., minutes, hours, days, months, years, or lifetime).

The terms “therapeutically effective amount” or “therapeutically effective dose” is the amount of a pharmaceutical composition sufficient to provide a beneficial effect to the individual to whom the pharmaceutical composition is administered. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

### Description

Complement activation is essential to a robust immune system. However, in certain instances, excessive or uncontrolled complement activation is a central component of numerous pathological conditions. Additionally, viral vector-based gene therapies are substantially hindered by unwanted immunological side-effects in which complement activation is involved. These immune responses can include antibody, B cell and/or T cell responses and can be specific to viral antigens of the viral vector, such as viral capsid or coat proteins or peptides thereof (see, e.g., Colella et al., 2018, Molecular Therapy: Methods & Clinical Development, 8:87-104).

The present invention arises in part from the inventors' development of proteinogenic compstatin analogs with robust complement inhibitory activity, the encoding nucleic acids of which can be introduced into a nucleic acid construct, such as a viral vector, and expressed in cells. These constructs can be used as stand-alone agents to provide long-acting gene therapy for treatment of complement activation-mediated disease, or inserted into viral vectors in combination with sequences encoding other therapeutic modalities (e.g., such as anti-VEGF agents). Combination of such proteinogenic compstatin molecules with other therapeutic modalities may provide added or synergistic therapeutic benefit in diseases driven by deregulated complement activation and aberrant neoangiogenic responses, exemplified for instance by elevated VEGF levels or enhanced VEGF receptor signaling. Such combinations can improve viral vector transduction, prevent C3 opsonization of capsids, and/or reduce or prevent immune responses (e.g., antibody, B cell and/or T cell responses).

### Peptides, Transgenes and Vectors:

Particular embodiments of the invention feature proteinogenic derivatives of the compstatin analog Cp40 (SEQ ID NO:3; described in WO2013/036778). Peptides of the instant disclosure are represented by the sequence shown below.

(SEQ ID NO: 4)
Xaal-[Cys-Ile-Trp-Gln-Asp-Trp-Gly-Xaa2-His-Arg-
Cys]-Xaa3-Xaa4

In the above sequence, Xaa1 is absent or comprises the dipeptide Tyr-Ile, Xaa2 is Ala or Glu, Xaa3 is absent or is Ile, and Xaa4 represents one, two or three Lys residues. The Cys residues form a disulfide bond, to form a cyclic peptide composed of the sequence within the brackets.

One embodiment is represented by the sequence:

(SEQ ID NO: 5)
Tyr-Ile-[Cys-Ile-Trp-Gln-Asp-Trp-Gly-Xaa2-His-Arg-
Cys]-Ile-Xaa4

Exemplary sequences of the present disclosure include Cp50 and Cp51, shown below:

Cp50:
(SEQ ID NO: 6)
Tyr-Ile-[Cys-Ile-Trp-Gln-Asp-Trp-Gly-Ala-His-Arg-
Cys]-Ile-Lys-Lys-Lys
Cp51:
(SEQ ID NO: 7)
Tyr-Ile-[Cys-Ile-Trp-Gln-Asp-Trp-Gly-Glu-His-Arg-
Cys]-Ile-Lys-Lys-Lys

### Other Exemplary Sequences Include:

(SEQ ID NO: 8)
Tyr-Ile-[Cys-Ile-Trp-Gln-Asp-Trp-Gly-Ala/Glu-His-
Arg-Cys]-Ile-Lys-Lys
(SEQ ID NO: 9)
Tyr-Ile-[Cys-Ile-Trp-Gln-Asp-Trp-Gly-Ala/Glu-His-
Arg-Cys]-Ile-Lys
(SEQ ID NO: 10)
Tyr-Ile-[Cys-Ile-Trp-Gln-Asp-Trp-Gly-Ala/Glu-His-
Arg-Cys]-Ile
(SEQ ID NO: 11)
Tyr-Ile-[Cys-Ile-Trp-Gln-Asp-Trp-Gly-Ala/Glu-His-
Arg-Cys]
(SEQ ID NO: 12)
[Cys-Ile-Trp-Gln-Asp-Trp-Gly-Ala/Glu-His-Arg-Cys]-
Ile-Lys-Lys-Lys
(SEQ ID NO: 13)
[Cys-Ile-Trp-Gln-Asp-Trp-Gly-Ala/Glu-His-Arg-Cys]-
Ile-Lys-Lys
(SEQ ID NO: 14)
[Cys-Ile-Trp-Gln-Asp-Trp-Gly-Ala/Glu-His-Arg-Cys]-
Ile-Lys
(SEQ ID NO: 15)
[Cys-Ile-Trp-Gln-Asp-Trp-Gly-Ala/Glu-His-Arg-Cys]-
Ile
(SEQ ID NO: 16)
[Cys-Ile-Trp-Gln-Asp-Trp-Gly-Ala/Glu-His-Arg-Cys]

It has been described previously that Cp40-KK and Cp40-KKK not only exhibit increased solubility as compared to unmodified Cp40, they also exhibit increased plasma and vitreous retention and enhanced C3-binding as compared to Cp40 and other Cp40-based analogs (see WO 2019/195712). In fact, Cp40-KKK was shown to display in vivo residence times equal to or exceeding three months following intravitreal administration. Notably, both Cp40-KK and Cp40-KKK analogs display markedly improved pharmacokinetic properties as compared to Cp40.

The exemplified proteinogenic peptides Cp50 and Cp51 have been found to have in vitro and in vivo properties similar to those of Cp40-KK and Cp40-KKK. This is surprising in view of the absence of the unnatural residues within the backbone and at the termini of Cp40. Table 1 below shows certain of these properties, as compared with Cp40 and other compstatin analogs.

| | | Solubility^{a} | | Hydrophobicity^{b} |
|---|---|---|---|---|
| Name | Sequence | DPBS(mg/mL) | K_{D} (nM) | Retention (min) |
| Cp05/POT- | Ac-Ile-[Cys-Val- | 3.5 | 12 | 5.09 |
| 4/APL-1 | 1MeTrp-Gln-Asp-Trp- | | | |
| (WO2007/ | Gly-Ala-His-Arg-Cys]- | | | |
| 062249) | Thr (SEQ ID NO: 2) | | | |
| Cp40/(WO2013/ | dTyr-Ile-[Cys-Val- | 0.8 | 0.5 | 4.73 |
| 036778) | 1MeTrp-Gln-Asp-Trp- | | | |
| | Sar-Ala-His-Arg-Cys]- | | | |
| | mIle (SEQ ID NO: 3) | | | |
| Cp50 | Tyr-Ile-[Cys-Ile-Trp- | >250 | 0.5 | |
| | Gln-Asp-Trp-Gly-Ala- | | | |
| | His-Arg-Cys]-Ile-Lys- | | | |
| | Lys-Lys (SEQ ID NO: 5) | | | |
| Cp51 | Tyr-Ile-[Cys-Ile-Trp- | >250 | 1 | |
| | Gln-Asp-Trp-Gly-Glu- | | | |
| | His-Arg-Cys]-Ile-Lys- | | | |
| | Lys-Lys (SEQ ID NO: 6) | | | |

^{a}Measured as OD (280 nm) at saturation; DPBS: Dulbecco's phosphate buffered saline.
^{b}Measured as retention time during HPLC analysis on a C18 column.

A nucleic acid encoding Cp50 (YICIWQDWGAHRCIKKK (SEQ ID NO:6)) is set forth below, showing all possible codons for each residue:

(SEQ ID NO: 17)
TAT/TAC - ATA/ATT/ATC - TGT/TGC - ATA/ATT/ATC -
TGG - CAG/CAA - GAT/GAC - TGG - GGG/GGA/GGT/GGC -
GCG/GCA/GCT/GCC - CAT/CAC - CGG/CGA/CGT/CGC -
TGT/TGC - ATA/ATT/ATC - AAG/AAA - AAG/AAA -
AAG/AAA

A nucleic acid encoding Cp51 (YICIWQDWGEHRCIKKK (SEQ ID NO:7)) is set forth below, showing all possible codons for each residue:

(SEQ ID NO: 18)
TAT/TAC - ATA/ATT/ATC -/ TGT/TGC - ATA/ATT/ATC -
TGG - CAG/CAA - GAT/GAC - TGG - GGG/GGA/GGT/GGC -
GAG/GAA - CAT/CAC - CGG/CGA/CGT/CGC - TGT/TGC -
ATA/ATT/ATC - AAG/AAA - AAG/AAA - AAG/AAA

The compstatin analogs of the present invention are produced by expression of nucleic acids encoding the peptides inserted into a vector. The vector may be non-viral or viral. In certain embodiments, the vector is a viral vector as discussed below. The vector includes one or more polypeptides encoding the compstatin analogs disclosed herein.

In certain embodiments, the compstatin analogs described herein are produced in advance (i.e., not in cells or tissues of a patient), and incorporated into pharmaceutical compositions for use in various combination therapies, as described herein. These compstatin analogs may be prepared by various synthetic methods of peptide synthesis via condensation of one or more amino acid residues, in accordance with conventional peptide synthesis methods.

Alternatively, because the compstatin analogs disclosed herein are composed of naturally occurring amino acids, they can be produced by expression of the encoding polynucleotides in a suitable prokaryotic or eukaryotic system. For example, a DNA construct may be inserted into a plasmid vector adapted for expression in a bacterial cell (such as E. coli) or a yeast cell (such as Saccharomyces cerevisiae), or into a baculovirus vector for expression in an insect cell or a viral vector for expression in a mammalian cell. Such vectors comprise the regulatory elements necessary for expression of the DNA in the host cell, positioned in such a manner as to permit expression of the DNA in the host cell. Such regulatory elements required for expression are well known in the art, and include promoter sequences, transcription initiation sequences and, optionally, enhancer sequences. The peptides produced by gene expression in a recombinant prokaryotic or eukaryotic system may be purified according to methods known in the art.

The compstatin analog polynucleotides and polypeptides described above are advantageous in several ways. For instance, as mentioned above, these compstatin analogs display complement inhibitory activity that rivals even the most potent of compstatin analogs. As such, complement inhibition can be achieved with lower doses. Moreover, these compstatin analogs are expected to possess the same or similar pharmacokinetic profiles as their similar counterparts, Cp40-KK and Cp40-KKK. Additionally, due to the small size of the encoding polynucleotides, they can be added to any vector and not take up an appreciable amount of vector space that might be needed for one or more transgenes of interest. This is particularly important for vectors having limited insertion space, such as AAV vectors. As such, vector lines can be constructed that possess the compstatin analog polynucleotides as a standard component, to be used advantageously to improve the efficacy of those vectors in delivering any gene therapy.

Thus, in one embodiment, a vector comprises a compstatin analog peptide and at least one insertion site for another polypeptide, e.g., for gene therapy. As recited in the definitions, such polypeptides are sometimes referred to herein as “transgenes.” In certain embodiments, the compstatin polypeptides and the other polypeptides are contained within a single vector, while in other embodiments they are contained on multiple vectors. If contained on multiple vectors, the vectors may be combined into a single pharmaceutical composition and administered together. Alternatively, the multiple vectors can be administered separately.

In certain embodiments, the compstatin polynucleotides are arranged with one or more other transgenes to produce a fusion protein, wherein the compstatin analog is linked to the other gene product. This arrangement may be advantageous in certain instances, for example where the linking confers a benefit to the compstatin analog, such as targeting to a specific tissue, cell, or location within a cell, or improving one or more other pharmacokinetic parameters.

In some embodiments, the compstatin analog is fused or conjugated to the N-terminus of the heterologous polypeptide, while in other embodiments it is fused or conjugated to the C-terminus of the heterologous polypeptide. In some embodiments, a fusion protein or protein conjugate includes a linker (e.g., a flexible linker) between the compstatin analog and the heterologous polypeptide. In some embodiments, a fusion protein or protein conjugate lacks a linker and the compstatin analog is fused or conjugated directly to the heterologous polypeptide. The linker may be of any sequence and length that allows each polypeptide to remain biologically active, e.g., not sterically hindered.

In certain instances, a fusion protein comprises a compstatin analog of the present invention and a VEGF-controlling gene product. Such fusion proteins are capable of binding with specificity to C3 and/or C3b in addition to vascular endothelial growth factor A (e.g., VEGF-A). In certain instances, a fusion protein reduces or inhibits angiogenesis, e.g., as compared with a reference polypeptide. In certain instances, a reference polypeptide comprises an antibody (or fragment thereof) known as ranibizumab (marketed as Lucentis®, Genentech)).

Vehicles for expressing compstatin polynucleotides of the invention comprise the regulatory elements necessary for expression of coding sequences of DNA in a host cell, positioned in such a manner as to permit expression of the DNA in the host cell. Expression control elements or sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and optionally, sequences that enhance secretion of the encoded product. The term “expression cassette” is sometimes used herein to describe the compstatin polynucleotides or other transgenes together with expression control elements for promoting and controlling their expression in host cells.

In certain embodiments, the compstatin polynucleotides of the invention and/or other transgenes are carried on viral vectors. Examples of viral vectors include, but are not limited to, retroviral vectors (e.g., Moloney murine leukemia virus (MMLV), Harvey murine sarcoma virus, murine mammary tumor virus, Rous sarcoma virus), adenoviral vectors, adeno-associated viral (AAV) vectors, SV40-type viral vectors, polyomaviral vectors, Epstein-Barr viral vectors, papilloma viral vectors, herpes viral vectors, vaccinia viral vectors, and polio viral vectors.

In certain embodiments, the selected vectors are viral vectors primarily used in gene therapy. The compstatin polynucleotides and/or other transgene may be incorporated into any type of viral vectors that are used in gene therapy, such as recombinant retroviruses, adenovirus, adeno-associated virus (AAV), and herpes simplex virus-1.

Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected nucleic acid can be inserted into a vector and packaged in retroviral particles using techniques known in the art. Protocols for the production of replication-deficient retroviruses are known in the art.

In some embodiments, the retrovirus is a lentivirus. Lentiviruses include human immunodeficiency viruses (HIV-I and HIV-2), simian immunodeficiency virus (SIV), feline immunodeficiency virus (FIV), equine infections anemia (EIA), and visna virus. Vectors derived from lentiviruses can achieve significant levels of nucleic acid transfer in vivo, and have been used for in vivo delivery to the eye (Campochiaro et al., 2017, Hum Gene Ther. 28:99-111).

Herpes simplex virus (HSV)-based viral vectors are also suitable for use as provided herein. Many replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb.

In some embodiments, the vector is an adenovirus vector. Adenovirus is a large family of viruses containing double stranded DNA. They replicate within the nucleus of a host cell, using the host cell machinery to synthesize viral RNA, DNA and proteins. Adenoviruses are known in the art to affect both replicating and non-replicating cells, to accommodate large transgenes, and to code for proteins without integrating into the host cell genome. The virus can be made replication-deficient by deleting select genes required for viral replication. The expendable non-replication-essential E3 region is also frequently deleted to allow additional room for a larger DNA insert. The adenovirus on which a viral vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (Manassas, VA). Non-group C adenoviruses, and even non-human adenoviruses, can be used to prepare replication-deficient adenoviral vectors.

In certain embodiments, recombinant AAV (rAAV) is the selected vector. AAV particles contain a linear, single-stranded AAV nucleic acid genome associated with an AAV capsid protein coat. AAV cannot replicate without a helper virus which can be an adenovirus, vaccinia or herpes virus. In absence of a helper virus, AAV inserts its genome in the host cell chromosome assuming a latent state. Subsequent infection by the helper virus rescues the latent integrated copy which then replicates to produce infectious viral progeny.

Recombinant AAV (rAAV) vectors are composed of a recombinant viral genome and capsid proteins. A rAAV genome is assembled from polynucleotides encoding the transgene(s), regulatory elements, and viral elements necessary for packaging the rAAV genome. Methods for construction of rAAV genomes are known in the art. An AAV expression vector may be composed of the AAV inverted terminal repeats (ITRs) flanking a restriction site for insertion of the transgene, either directly using the restriction site available, or by excision of the transgene with restriction enzymes followed by polishing the ends and ligation into the AAV expression vector, optionally using linkers. A transgene is integrated in the AAV expression vector along with one or more expression control elements as described above, including, for example an enhancer, promoter, and/or a post transcriptional regulatory sequence (PRE), flanked by AAV ITRs.

Methods for making rAAV vectors having a specific capsid protein are known in the art. Briefly, viral particles are made by providing the components required for packaging the rAAV genome in a capsid in trans, or required components may be provided by an engineered host cell. Some or all of the required elements can be either under the control of an inducible or a constitutive promoter. The recombinant AAV genome, rep sequences, cap sequences, and helper functions for producing the rAAV may be delivered to the packaging host cell using any appropriate genetic element (vector). Typically, the recombinant AAVs are produced by transfecting a host cell with a recombinant AAV genome (containing a transgene) to be packaged into AAV particles, an AAV helper function vector, and an accessory function vector. An AAV helper function vector encodes the AAV helper function sequences (i.e., rep and cap), which function in trans for productive AAV replication and encapsidation. The accessory function vector typically encodes the nucleotide sequences for non-AAV derived viral and/or cellular functions that are required for AAV replication including those elements involved in activation of AAV gene transcription, stage specific AAV mRNA splicing, AAV DNA replication, synthesis of cap expression products, and AAV capsid assembly.

The AAV vectors described herein generally comprise a rAAV genome encoding one or more transgenes operably linked to one or more regulatory elements in a manner that permits transgene transcription, translation, and/or expression in a target cell or a target tissue and is flanked by 5′ and 3′ ITRs. ITR sequences are typically about 145 bp in length. The AAV ITR sequences can be modified, e.g., by the insertion, deletion or substitution of one or more nucleotides by using standard molecular biology techniques, as long as the modification of the ITR sequence does not interfere with AAV vector function. AAV ITRs may be derived from any of the several AAV serotypes. The AAV ITR sequences at 3′ and 5′ can be identical or derived from different AAV serotype.

Any appropriate AAV serotype or combination of AAV serotypes can be used in the methods and compositions described herein. Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. In some embodiments, an AAV vector is an AAV2/6, AAV2/8 or AAV2/9 vector (e.g., AAV6, AAV8 or AAV9 serotype having AAV2 ITR). Other AAV vectors are described in, e.g., Sharma et al., Brain Res Bull. 2010 Feb. 15; 81 (2-3): 273. Generally, any AAV serotype may be used to deliver a transgene described herein. However, the serotypes have different tropisms, e.g., they preferentially infect different tissues. In certain embodiments, AAV serotypes that target a cell type or organ of interest can be employed. For example, serotypes targeting the central nervous system (among other targets) include but are not limited to AAV1, AAV2, AAV4, AAV5, AAV6, AAV7, AAV8, AAV9, and AAV10. As another example, favorable retinal gene transfer has been observed with serotypes AAV1, AAV2, AAV4, AAV5, AAV7, AAV8 and AAV9. AAV7 and AAV8 have been shown to enable superior long-term transduction of retinal and also anterior chamber structures. (Lebherz C, Maguire A, Tang W, Bennett J, Wilson J M. J Gene Med. 2008 April; 10 (4): 375-82. doi: 10.1002/jgm.1126.PMID: 18278824).

A promoter operably linked to a transgene can either be inducible or constitutive. Inducible promoters allow regulation of gene expression and can be regulated by exogenously circumstances or compounds. Constitutive promoters are unregulated promoter that allows for continual transcription of its associated gene. Examples of inducible promoters include a zinc-inducible sheep metallothionine (MT) promoter, a dexamethasone (Dex)-inducible mouse mammary tumor virus (MMTV) promoter, a T7 polymerase promoter system; an ecdysone inducible promoter system, and a tetracycline-repressor inducible system. Examples of constitutive promoters include a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter and a 3-actin promoter. In certain embodiments, a native promoter or fragment thereof for a selected transgene may be used if the expression of the transgene to mimic the native expression is preferred.

In some embodiments, regulatory sequences impart tissue-specific gene expression capabilities. Such tissue-specific regulatory sequences (e.g., promoters, enhancers, etc.) are known in the art. For instance, promoters that are active in liver include the transthyretin (TTR) gene promoter; human alpha 1-antitrypsin (hAAT) promoter; the lipoprotein A-I promoter; hepatitis B virus core promoter; alpha-fetoprotein (AFP); human Factor IX promoter; thyroxin binding globulin (TBG) promoter; TTR minimal enhancer/promoter, alpha-antitrypsin promoter; and LSP1 promoter. An example of an enhancer active in liver is apolipoprotein E (ApoE) hepatic control region 1 (HCR-1) and 2 (HCR-2). As another example, suitable neuronal specific promoters include, but are not limited to, neuron specific enolase (NSE) (GenBank Accession No: X51956), and human neurofilament light chain promoter (NEFL) (GenBank Accession No: L04147). Glial specific promoters include, but are not limited to, glial fibrillary acidic protein (GFAP) promoter (GenBank Accession No: M65210), S100 promoter (GenBank Accession No: M65210) and glutamine synthase promoter (GenBank Accession No: X59834).

The transgene of a gene therapy viral vector as described herein may encode a functional version of any protein that, through some defect in the endogenous version in a subject (including a defect in the expression of an endogenous version), results in a disease or disorder in the subject.

Therapeutic proteins also include Myophosphorylase, glucocerebrosidase, fibroblast growth factor receptor 3, huntingtin, HFE protein, CFTR, frataxin, VMD2, hemoglobin, phenylalanine hydroxylase, fibrillin, dystophia myotonica protein kinase, lignoceroyl-CoA ligase, dystrophin, methylCpG-binding protein 2, Beta hemoglobin, Myotubularin, Cathepsin A, Factor IX, Lipoprotein lipase, Beta galactosidase, Ornithine Transcarbamylase, Iduronate-2-Sulfatase, Acid-Alpha Glucosidase, UDP-glucuronosyltransferase 1-1, GlcNAc-1-phosphotransferase, GlcNAc-1-phosphotransferase, Mucolipin-1, Microsomal triglyceride transfer protein, Sphingomyelinase, Acid ceramidase, Lysosomal acid lipase, Alpha-L-iduronidase, Heparan N-sulfatase, alpha-N-acetylglucosaminidase, acetyl-CoA alpha-glucosaminide acety1transferase, N-acetylglucosamine 6-sulfatase, N-acetylgalactosamine-6 sulfatase, Alpha-mannosidase, Alpha-galactosidase A, Cystic fibrosis conductance transmembrane regulator, and respiratory proteins

As further examples, therapeutic proteins also include functional versions of proteins associated with disorders of lipid and sphingolipid degradation (e.g., beta-Galactosidase-1, beta-Hexosaminidases A and B, GM2 Activator Protein, 8-Galactosidase A, Glucocerebrosidase, Glucocerebrosidase, Glucocerebrosidase, Arylsulfatase A, Galactosylceramidase, Sphingomyelinase, Sphingomyelinase, NPCI, HEI protein (Cholesterol Trafficking Defect), Acid Ceramidase, Lysosomal Acid Lipase); disorders of mucopolysaccharide degradation (e.g., L-Iduronidase, L-Iduronidase, L-Iduronidase, Iduronate Sulfatase, Heparan N-Sulfatase, N-Acetylglucosaminidase, Acetyl-CoA-Glucosaminidase, Acetyltransferase, Acetylglucosamine-6-SulFAtase, Galactosamine-6-SulFAtase, Arylsulfatase B, Glucuronidase); disorders of glycoprotein degradation (e.g., Mannosidase, mannosidase, 1-fucosidase, Aspartylglycosaminidase, Neuraminidase, Lysosomal protective protein, Lysosomal 8-N-acetylgalactosaminidase, Lysosomal 8-N-acetylgalactosaminidase); lysosomal storage disorders (e.g., Palmitoyl-protein thioesterase, at least 4 subtypes, Lysosomal membrane protein, Glucose-6-phosphatase, Glucose-6-phosphate translocase, Acid maltase, Debrancher enzyme amylo-1,6 glucosidase, N-acetylglucosamine-1-phosphotransferase, N-acetylglucosamine-1-phosphotransferase, Ganglioside sialidase (neuraminidase), Lysosomal cystine transport protein, Lysosomal cystine transport protein, Lysosomal cystine transport protein, Sialic acid transport protein Saposins, A, B, C, D) and leukodystrophies (e.g., Microsomal triglyceride transfer protein/apolipoprotein B, Peroxisomal membrane transfer protein, Peroxins, Aspartoacylase, Sterol-27-hydroxlase, Proteolipid protein, ABCI transporter, Peroxisome membrane protein 3 or Peroxisome biogenesis factor 1, Phytanic acid oxidase).

The viral vectors described herein may be used for gene editing. In such embodiments, the transgene of the viral vector is a gene editing transgene. Such a transgene encodes an agent or component that is involved in a gene editing process. Generally, such a process results in long-lasting or permanent modifications to genomic DNA, such as targeted DNA insertion, replacement, mutagenesis or removal. Gene editing may include the delivery of nucleic acids encoding a DNA sequence of interest and inserting the sequence of interest at a targeted site in genomic DNA using endonucleases. Thus, gene editing transgenes may comprise these nucleic acids encoding a DNA sequence of interest for insertion. In some embodiments, the DNA sequence for insertion is a DNA sequence encoding any one of the therapeutic proteins described herein. Additionally or alternatively, the gene editing transgene may comprise nucleic acids that encode one of more components that can alone or in combination with other components carry out the gene editing process, as known in the art.

The viral vectors described herein may be used for gene expression modulation. In such embodiments, the transgene of the viral vector is a gene expression modulating transgene. Such a transgene encodes a gene expression modulator that can enhance, inhibit (e.g., silence) or modulate the expression of one or more endogenous genes. The endogenous gene may encode any one of the proteins as described herein provided the protein is an endogenous protein of the subject. Accordingly, the subject may be one with any one of the diseases or disorders described herein where there would be a benefit provided by gene expression modulation.

Gene expression modulators include DNA-binding proteins (e.g., artificial transcription factors and transcriptional silencer protein NRF), as well as therapeutic RNAs. Therapeutic RNAs include inhibitors of mRNA translation (antisense), agents of RNA interference (RNAi), catalytically active RNA molecules (ribozymes) and RNAs that bind proteins and other molecular ligands (aptamers), among others.

Exemplary transgenes encode interfering RNA, antisense RNA, ribozymes, and aptamers that decrease the level of an angiogenic factor in a cell. For example, an RNAi can be a miRNA, an shRNA, or an siRNA that reduces the level of vascular endothelial growth factor (VEGF) in a cell. For example, an RNAi can be an shRNA or siRNA that reduces the level of VEGF or VEGF receptor (VEGFR) in a cell. RNAi agents that target VEGF include, e.g., an RNAi described in U.S. Patent Publication No. 2011/0224282. For example, an siRNA specific for VEGF-A, VEGFR1, or VEGFR2 would be suitable. Suitable nucleic acid gene products also include a ribozyme specific for VEGF-A, VEGFR1, or VEGFR2; an antisense specific for VEGF-A, VEGFR1, or VEGFR2; siRNA specific for VEGF-A, VEGFR1, or VEGFR2; and the like. Also suitable as a gene product is an miRNA that reduces the level of VEGF by regulating VEGF gene expression, e.g., through post-transcriptional repression or mRNA degradation. Examples of suitable miRNA include, e.g., miR-15b, miR-16, miR-20a, and miR-20b. See, e.g., Hua et al. (2006) PLOS ONE 1: e116. Also suitable is an anti-VEGF aptamer (e.g., EYEOOI) For anti-VEGF aptamers, see, e.g., Ng et al. (2006) Nature Reviews Drug Discovery 5:123; and U.S. Pat. Nos. 6,426,335; 6,168,778; 6,147,204; 6,051,698; and 6,011,020. For example, an aptamer can be directed against VEGF16s, the isoform primarily responsible for pathological ocular neovascularization and vascular permeability.

In some embodiments, a transgene encodes a polypeptide (e.g., an antibody or fusion protein) that inhibits or reduces activity of a disease-causing or disease-promoting polypeptide. For example, in some embodiments, a transgene encodes an anti-angiogenic polypeptide including, e.g., vascular endothelial growth factor (VEGF) antagonists. Suitable VEGF antagonists include, but are not limited to, inhibitors of VEGFR1 tyrosine kinase activity; inhibitors of VEGFR2 tyrosine kinase activity; an antibody to VEGF; an antibody to VEGFR1; an antibody to VEGFR2; a soluble VEGFR; and the like. Antibodies specific for VEGF include, e.g., bevacizumab (AVASTIN™) and ranibizumab (also known as rhuFAb V2). Also suitable for use are anti-angiogenic polypeptides such as endostatin, PEDF, and angiostatin.

Anti-angiogenic polypeptides include, e.g., recombinant polypeptides comprising VEGF receptors. For example, a suitable anti-angiogenic polypeptide can be the soluble form of the VEGFR-1, known as sFlt-1 (Kendall et al. (1996) Biochem. Biophys. Res Commun. 226:324). Suitable anti-angiogenic polypeptides also include an immunoglobulin-like (lg) domain 2 of a first VEGF receptor (e.g., Flt1), alone or in combination with an lg domain 3 of a second VEGF receptor (e.g., Flk1 or Flt4); an anti-angiogenic polypeptide can also include a stabilization and/or a multimerization component. Such recombinant anti-angiogenic polypeptides are described in, e.g., U.S. Pat. No. 7,521,049. Anti-VEGF antibodies that are suitable as heterologous gene products include single chain Fv (scFv) antibodies. See, e.g., U.S. Pat. Nos. 7,758,859; and 7,740,844, for anti-VEGF antibodies. Additional transgenes are described in, e.g., Bordet et al., Drug Discov Today. 2019 Jun. 5. pii: S1359-6446 (18) 30472-0 doi: 10.1016/j.drudis.2019.05.038. Such transgenes can be used to treat ocular disorders, e.g., age-related macular degeneration.

In certain embodiments, the compstatin analogs described herein are produced in advance (i.e., not in cells or tissues of a patient), and incorporated into pharmaceutical compositions for use in various combination therapies, as described herein. These compstatin analogs may be prepared by various synthetic methods of peptide synthesis via condensation of one or more amino acid residues, in accordance with conventional peptide synthesis methods.

Alternatively, inasmuch as the compstatin analogs disclosed herein are composed of naturally occurring amino acids, they can be produced by expression of the encoding polynucleotides in a suitable prokaryotic or eukaryotic system. For example, a DNA construct may be inserted into a plasmid vector adapted for expression in a bacterial cell (such as E. coli) or a yeast cell (such as Saccharomyces cerevisiae), or into a baculovirus vector for expression in an insect cell or a viral vector for expression in a mammalian cell. Such vectors comprise the regulatory elements necessary for expression of the DNA in the host cell, positioned in such a manner as to permit expression of the DNA in the host cell. Such regulatory elements required for expression are well known in the art, and include promoter sequences, transcription initiation sequences and, optionally, enhancer sequences. The peptides produced by gene expression in a recombinant prokaryotic or eukaryotic system may be purified according to methods known in the art.

### Pharmaceutical Compositions and Administration Thereof:

To implement one or more of the utilities mentioned herein, another aspect of the invention features pharmaceutical compositions comprising vectors harboring the compstatin analog polynucleotides described and exemplified herein. Such a pharmaceutical composition may include the active ingredient (e.g. viral vector(s) containing transgenes), in a form suitable for administration to a subject, or the pharmaceutical composition may comprise the active ingredient and one or more pharmaceutically acceptable carriers, one or more additional ingredients, or some combination of these. These pharmaceutical compositions can be delivered to a subject, so as to allow production of the polypeptides and encoded protein(s). In certain embodiments, pharmaceutical compositions comprise sufficient genetic material to enable a recipient to produce a therapeutically effective amount of a protein in the subject.

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration can be proportionally increased or reduced, as indicated by any adverse side effects, complications or other risk factors of the treatment or therapy and the status of the subject. The skilled artisan will appreciate the factors that can influence the dosage and timing required to provide an amount sufficient for providing a therapeutic or prophylactic benefit.

The dose to achieve a therapeutic effect, e.g., the dose in vector genomes/per kilogram of body weight (vg/kg) of a viral vector, such as rAAV, will vary based on several factors including, but not limited to: route of administration, choice of AAV vector serotype, viral transduction efficiency in selected tissues of interest, the level of heterologous polynucleotide expression required to achieve a therapeutic effect, the specific disease treated, any host immune response to the viral vector, a host immune response to the heterologous polynucleotide or expression product (protein), and/or the stability of the protein expressed. One skilled in the art can determine a vector genome or non-viral vector dose range to treat a patient having a particular disease or disorder based on the aforementioned factors, as well as other factors.

In embodiments utilizing rAAV vectors, viral vector doses will range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. In particular, dose ranges of recombinant AAV vg/kg are from about 1×10¹¹ to about 5×10¹³ vg/kg, and within that range up to about 2×10¹¹, or about 3×10¹¹, or about 4×10¹¹, or about 5×10¹¹, or about 6×10¹¹, or about 7×10¹¹, or about 8×10¹¹, or about 9×10¹¹, or about 1×10¹², or about 2×10¹², or about 3×10¹², or about 4×10¹², or about 5×10¹², or about 6×10¹², or about 7×10¹², or about 8×10¹², or about 9×10¹², or about 1×10¹³, or about 2×10¹³, or about 3×10¹³, or about 4×10¹³ recombinant AAV vg/kg.

A “dosage unit” as used herein refers to physically discrete units suited as unitary dosages for the subject to be treated; each unit containing a predetermined quantity optionally in association with a pharmaceutical carrier which, when administered in one or more doses, is calculated to produce a desired effect (e.g., prophylactic or therapeutic effect). In various embodiments, dosage units can be contained within ampules and vials, which can include a liquid composition, or a composition in a freeze-dried or lyophilized state; a sterile liquid carrier, for example, can be added prior to administration or delivery in vivo. Individual dosage units can be included in multi-dose kits or containers. Viral particles, non-viral vectors, and pharmaceutical compositions thereof can be packaged in single or multiple unit dosage form for ease of administration and uniformity of dosage.

In certain embodiments, dosages are calculated based on the amount of therapeutic gene product produced in target cells or tissues of a subject. As stated above, such dosages will vary based, among other things, on the gene product(s) and the disease being treated. However, inasmuch as the compstatin analogs of the disclosure are produced in various embodiments, dosage of these peptides can be addressed.

The pharmaceutical compositions useful for practicing the invention may be administered to deliver an amount of vector sufficient to produce a dose of compstatin analog at between about 0.0005 mg/kg and 50 mg/kg body weight. In certain embodiments, the dosage comprises at least 0.0005 mg/kg, or at least 0.001 mg/kg, or at least 0.002 mg/kg, or at least 0.005 mg/kg, or at least 0.01 mg/kg, or at least 0.02 mg/kg, or at least 0.04 mg/kg, or at least 0.05 mg/kg, or at least 0.1 mg/kg, or at least 0.2 mg/kg, or at least 0.3 mg/kg, or at least 0.4 mg/kg, or at least 0.5 mg/kg, or at least 0.6 mg/kg, or at least 0.7 mg/kg, or at least 0.8 mg/kg, or at least 0.9 mg/kg, or at least 1 mg/kg, or at least 2 mg/kg, or at least 3 mg/kg, or at least 4 mg/kg (14.9 μM), or at least 5 mg/kg, or at least 6 mg/kg, or at least 7 mg/kg, or at least 8 mg/kg, or at least 9 mg/kg, or at least 10 mg/kg, or at least 15 mg/kg, or at least 20 mg/kg, or at least 25 mg/kg, or at least 30 mg/kg, or at least 35 mg/kg, or at least 40 mg/kg, or at least 45 mg/kg, or at least 50 mg/kg, on a daily basis or on another suitable periodic regimen.

In one embodiment, the invention envisions intravenous or subcutaneous administration of a vector containing a compstatin analog polynucleotide, as described herein, to produce in target cells or tissues a dose of compstatin analog that is between about 0.0005 mg/kg and about 10 mg/kg, e.g., 0.0005 mg/kg, 0.001 mg/kg, 0.002 mg/kg, 0.005 mg/kg, 0.01 mg/kg, 0.02 mg/kg, 0.04 mg/kg, 0.05 mg/kg, 0.1 mg/kg, 0.125 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg (14.9 μM), 2.25 mg/kg, 2.5 mg/kg, 2.75 mg/kg, 3 mg/kg, 3.25 mg/kg, 3.5 mg/kg, 3.75 mg/kg, 4 mg/kg, 4.25 mg/kg, 4.5 mg/kg, 4.75 mg/kg, 5 mg/kg, 5.25 mg/kg, 5.5 mg/kg, 5.75 mg/kg, 6 mg/kg, 6.25 mg/kg, 6.5 mg/kg, 6.75 mg/kg, 7 mg/kg, 7.25 mg/kg, 7.5 mg/kg, 7.75 mg/kg, 8 mg/kg, 8.25 mg/kg, 8.5 mg/kg, 8.75 mg/kg, 9 mg/kg, 9.25 mg/kg, 9.5 mg/kg, 9.75 mg/kg, or 10 mg/kg. In certain embodiments, vectors are administered via intravenous or subcutaneous delivery (e.g., injection or infusion) to produce a dose of compstatin analog that is between about 0.25 mg/kg and about 5 mg/kg. In another embodiment, the dose is between about 0.5 mg/kg and about 5 mg/kg. In yet another embodiment, the dose is between about 0.5 mg/kg and 4 mg/kg or between about 0.5 mg/kg and about 3 mg/kg.

In another embodiment, the invention envisions intramuscular administration of a vector containing a compstatin analog polynucleotide to produce in target cells or tissues dose that is between about 0.0005 mg/kg and about 50 mg/kg, e.g., 0.0005 mg/kg, 0.001 mg/kg, 0.002 mg/kg, 0.005 mg/kg, 0.01 mg/kg, 0.02 mg/kg, 0.04 mg/kg, 0.05 mg/kg, 0.125 mg/Kg, 0.25 mg/kg, 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, or 50 mg/kg. In a preferred embodiment, vectors are administered via intramuscular delivery (e.g., injection) in an amount sufficient to produce a dose of compstatin analog that is between about 0.25 mg/kg and about 35 mg/kg. In another embodiment, the dose is between about 0.25 mg/kg and 30 mg/kg; or between about 0.25 mg/kg and 10 mg/kg; or between about 0.25 mg/kg and 5 mg/kg. For instance, in one particular embodiment, the intended dose of compstatin analog is about 2.5 mg/kg.

In yet another embodiment, vectors are introduced by intravitreal administration to produce a dose of compstatin analog in the vitreous of an eye that is between about 1 μg and about 10 mg (931 μM), e.g., 1 μg, 1.25 μg, 1.5 μg, 1.75 μg, 2 μg, 2.25 μg, 2.5 μg, 2.75 μg, 3 μg, 3.25 μg, 3.5 μg, 3.75 μg, 4 μg, 4.25 μg, 4.5 μg, 4.75 μg, 5 μg, 5.25 μg, 5.5 μg, 5.75 μg, 6 μg, 6.25 μg, 6.5 μg, 6.75 μg, 7 μg, 7.25 μg, 7.5 μg, 7.75 μg, 8 μg, 8.25 μg, 8.5 μg, 8.75 μg, 9 μg, 9.25 μg, 9.5 μg, 9.75 μg, 10 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg 900 μg, 950 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg; preferably, the dose is between about 1 μg and about 2,000 μg, e.g., about 10 μg to about 1,800 μg or about 100 μg to about 1,500 μg, or about 500 μg to about 1,200 μg, or about 500 μg to about 1,000 μg. In some embodiments, the therapeutically effective dose of compstatin analog in the vitreous is at least about 0.02 mg, e.g., at least about 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85, mg, 0.9 mg, 0.95 mg, or 1 mg. Similar doses can be used for subretinal delivery.

In another embodiment, pharmaceutical compositions containing compstatin or its encoding polypeptides can be delivered to the central nervous system, using dosing ranges within the ranges described herein. For example, research has shown that intraparenchymal rAAV injections can result in localized distribution of rAAV and are suitable for the treatment of CNS diseases that afflict a defined region of the brain, such as the putamen in Parkinson disease. As another example, delivery to the cerebrospinal fluid space by intrathecal injection can achieve broader CNS distribution. (see, Nat Rev Drug Discov. 2019 May; 18 (5): 358-378).

In another embodiment, the invention envisions oral administration of pharmaceutical compositions described herein. While the oral route is not typical for vector-based gene therapy, it can be suitable for combination therapy involving administration of peptides by one route and administration of vectors by another route. Here the dosage of peptide ranges between about 1 mg/kg and about 20 mg/kg, e.g., 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, or 20 mg/kg. In a preferred embodiment, compstatin analog peptides are administered via oral delivery at a therapeutically effective dose that is between about 1 mg/kg and about 10 mg/kg. For instance, in one particular embodiment, peptides are delivered orally to a human at a dose of about 1 and 5 mg/kg. In some embodiments, the oral dose described herein is administered once. In other embodiments, it is administered periodically, e.g., daily.

In another embodiment, the invention envisions periodontal administration, such as intrapapillary infiltration, of vectors encoding compstatin analogs, as described herein, at a dose that is between about 1 μg and about 1,000 μg, e.g., 1 μg, 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 550 μg, 600 μg, 650 μg, 700 μg, 750 μg, 800 μg, 850 μg, 900 μg, 950 μg, or 1,000 μg. For example, vectors can be administered periodontally to a human to result in a dose of compstatin analog of between about 5 μg and about 500 μg. In a particular embodiment, vectors are administered periodontally to a human to produce a dose of compstatin analog of between about 10 μg/interdental papilla and about 200 μg/interdental papilla or at a dose of between about 20 μg/interdental papilla and about 100 μg/interdental papilla.

In one embodiment, vectors are administered in an amount resulting in a serum concentration of the compstatin analogs of between about 0.01 nM and about 30 μM in an individual. In certain embodiments, the combined dose and regimen will result in a serum concentration, or an average serum concentration over time, of the compstatin analogs of at least about 0.01 nM, or at least about 0.02 nM, or at least about 0.03 nM, or at least about 0.04 nM, or at least about 0.05 nM, or at least about 0.06 nM, or at least about 0.07 nM, or at least about 0.08 nM, or at least about 0.09 nM, or at least about 0.1 nM, 0.11 nM, or at least about 0.12 nM, or at least about 0.13 nM, or at least about 0.14 nM, or at least about 0.15 nM, or at least about 0.16 nM, or at least about 0.17 nM, or at least about 0.18 nM, or at least about 0.19 nM, or at least about 0.2 nM, or at least about 0.3 nM, or at least about 0.4 nM, or at least about 0.5 nM, or at least about 0.6 nM, or at least about 0.7 nM, or at least about 0.8 nM, or at least about 0.9 nM, or at least about 1 nM or at least about 1.5 nM, or at least about 2 nM, or at least about 2.5 nM, or at least about 3 nM, or at least about 3.5 nM, or at least about 4 nM, or at least about 4.5 nM, or at least about 5 nM, or at least about 5.5 nM, or at least about 6 nM, or at least about 6.5 nM, or at least about 7 nM, or at least about 7.5 nM, or at least about 8 nM, or at least about 8.5 nM, or at least about 9 nM, or at least about 9.5 nM, or at least about 10 nM (0.01 μM).

In certain embodiments, the combined dose and regimen will result in a serum concentration, or an average serum concentration over time, of the compstatin analogs of at least about 0.01 μM, or at least about 0.02 μM, or at least about 0.03 μM, or at least about 0.04 μM, or at least about 0.05 μM, or at least about 0.06 μM, or at least about 0.07 μM, or at least about 0.08 μM, or at least about 0.09 μM, or at least about 0.1 μM, 0.11 μM, or at least about 0.12 μM, or at least about 0.13 μM, or at least about 0.14 μM, or at least about 0.15 μM, or at least about 0.16 μM, or at least about 0.17 μM, or at least about 0.18 μM, or at least about 0.19 μM, or at least about 0.2 μM, or at least about 0.3 μM, or at least about 0.4 μM, or at least about 0.5 μM, or at least about 0.6 μM, or at least about 0.7 μM, or at least about 0.8 μM, or at least about 0.9 μM, or at least about 1 μM or at least about 1.5 μM, or at least about 2 μM, or at least about 2.5 μM, or at least about 3 μM, or at least about 3.5 μM, or at least about 4 μM, or at least about 4.5 μM, or at least about 5 μM, or at least about 5.5 μM, or at least about 6 μM, or at least about 6.5 μM, or at least about 7 μM, or at least about 7.5 μM, or at least about 8 μM, or at least about 8.5 μM, or at least about 9 μM, or at least about 9.5 μM, or at least about 10 μM, or at least about 10.5 μM, or at least about 11 μM or at least about 11.5 μM, or at least about 12 μM, or at least about 12.5 μM, or at least about 13 μM, or at least about 13.5 μM, or at least about 14 μM, or at least about 14.5 μM, or at least about 15 μM, or at least about 15.5 μM, or at least about 16 μM, or at least about 16.5 μM, or at least about 17 μM, or at least about 17.5 μM, or at least about 18 μM, or at least about 18.5 μM, or at least about 19 μM, or at least about 19.5 μM, or at least about 20 μM, or at least about 20.5 μM, or at least about 21 μM or at least about 21.5 μM, or at least about 22 μM, or at least about 22.5 μM, or at least about 23 μM, or at least about 23.5 M, or at least about 24 μM, or at least about 24.5 μM, or at least about 25 μM, or at least about 25.5 μM, or at least about 26 μM, or at least about 26.5 μM, or at least about 27 μM, or at least about 27.5 μM, or at least about 28 μM, or at least about 28.5 μM, or at least about 29 μM, or at least about 29.5 μM, or at least about 30 μM. In certain embodiments, the combined dose and regimen will result in a serum concentration, or an average serum concentration over time, of the compstatin analogs of up to about 0.1 μM, or up to about 0.11 μM, or up to about 0.12 μM, or up to about 0.13 μM, or up to about 0.14 μM, or up to about 0.15 μM, or up to about 0.16 μM, or up to about 0.17 μM, or up to about 0.18 μM, or up to about 0.19 μM, or up to about 0.2 μM, or up to about 0.3 μM, or up to about 0.4 μM, or up to about 0.5 μM, or up to about 0.6 μM, or up to about 0.7 μM, or up to about 0.8 μM, or up to about 0.9 μM, or up to about 1 μM or up to about 1.5 μM, or up to about 2 μM, or up to about 2.5 μM, or up to about 3 μM, or up to about 3.5 μM, or up to about 4 μM, or up to about 4.5 μM, or up to about 5 μM, or up to about 5.5 μM, or up to about 6 μM, or up to about 6.5 μM, or up to about 7 μM, or up to about 7.5 M, or up to about 8 μM, or up to about 8.5 M, or up to about 9 μM, or up to about 9.5 μM, or up to about 10 μM, or up to about 10.5 μM or up to about 11 μM or up to about 11.5 μM, or up to about 12 μM, or up to about 12.5 μM, or up to about 13 μM, or up to about 13.5 μM, or up to about 14 μM, or up to about 14.5 μM, or up to about 15 μM, or up to about 15.5 μM, or up to about 16 μM, or up to about 16.5 μM, or up to about 17 μM, or up to about 17.5 μM, or up to about 18 μM, or up to about 18.5 μM, or up to about 19 μM, or up to about 19.5 μM, or up to about 20 μM, or up to about 20.5 μM or up to about 21 μM or up to about 21.5 μM, or up to about 22 μM, or up to about 22.5 μM, or up to about 23 μM, or up to about 23.5 μM, or up to about 24 μM, or up to about 24.5 μM, or up to about 25 μM, or up to about 25.5 M, or up to about 26 μM, or up to about 26.5 μM, or up to about 27 μM, or up to about 27.5 μM, or up to about 28 μM, or up to about 28.5 μM, or up to about 29 μM, or up to about 29.5 μM, or up to about 30 μM.

Suitable ranges include about 0.1 to about 30 μM, or about 1 to about 29 μM, or about 2 to about 28 μM, or about 3 to about 27 μM, or about 4 to about 26 μM, or about 5 to about 25 μM, or about 6 to about 24 μM, or about 7 to about 23 μM, or about 8 to about 22 μM, or about 9 to about 21 μM, or about 10 to about 20 μM, or about 11 to about 19 μM, or about 12 to about 18 μM, or about 13 to about 17 μM, or about 1 to about 5 μM, or about 5 to about 10 μM, or about 10 to about 15 μM, or about 15 to about 20 μM, or about 20 to about 25 μM, or about 25 to about 30 μM. While the precise dosage administered will vary depending upon any number of factors, including but not limited to, the type of patient and type of disease state being treated, the age of the patient and the route of administration, such dosage is readily determinable by the person of skill in the art.

As such, in particular embodiments, pharmaceutical compositions comprising the compstatin analog transgenes and/or other transgenes and/or natural amino acid-containing compstatins produced by other means and derivatives with other therapeutic molecules are administered via subcutaneous, intradermal, intravenous, intraocular (including intravitreal, subretinal), intracerebral, intraperitoneal, intramuscular injection, periodontal administration (including gingival administration or intrapapillary infiltration injection), intranasal, epidural, oral, sublingual, intrathecal, intravaginal, transdermal, rectal, by inhalation, or topical administration.

The formulations of the pharmaceutical compositions may be prepared by any method known or hereafter developed in the art of pharmaceutical technology. In general, such preparatory methods include the step of bringing the active ingredient into association with a carrier or one or more other accessory ingredients, and then, if necessary or desirable, shaping or packaging the product into a desired single- or multi-dose unit.

The pharmaceutical composition described herein can be administered to a patient as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the patient, as described above. However, and as noted above, vectors producing the compstatin analogs of the instant disclosure can be administered at less frequent intervals as compared to previously known compstatin analogs.

For instance, in some embodiments, the intravenous, intramuscular, intraocular (including intravitreal), subcutaneous, periodontal (including gingival administration or intrapapillary infiltration) or topical administration of a pharmaceutical composition containing the presently described vectors is via a single injection. Additionally, and given the extended residence time of the presently described compstatin analogs, certain embodiments envision long term systemic administration of these compstatin analog polynucleotides, for instance by intravenous, intraocular (including intravitreal and subretinal), subcutaneous, intramuscular, periodontal (including gingival administration or intrapapillary infiltration) or topical administration routes at the above-described therapeutic doses via multiple deliveries over time in order to provide a therapeutically effective maintenance dose of the compstatin analogs and other gene therapy, depending on the type and age of patient and the type and severity of disease treated. Thus, in some embodiments, vectors are delivered once every about 12 hours to about once every three months or even once every 5-6 months, e.g., once every 12 hours, once every 24 hours, once every 2 days, once every 3 days, once every 4 days, once every 5 days, once every 6 days, once every 7 days, once every 8 days, once every 9 days, once every 10 days, once every 2 weeks, once every 3 weeks, once every month, once every two months, once every three months or once every 5-6 months. In other embodiments, vectors are delivered once every about 12 hours to about once every three months, e.g., once every 12 hours, once every 24 hours, once every 2 days, once every 3 days, once every 4 days, once every 5 days, once every 6 days, once every 7 days, once every 8 days, once every 9 days, once every 10 days, once every 2 weeks, once every 3 weeks, once every month, once every two months, once every three months.

As noted above, pharmaceutical compositions containing the compstatin analog and/or other gene therapy encoding vectors and/or natural amino acid-containing compstatins produced by any other means and derivatives with other therapeutic molecules may be formulated for administration by a variety of routes. Such pharmaceutical compositions may contain pharmaceutically acceptable carriers and other ingredients known to enhance and facilitate drug administration. Other formulations, such as nanoparticles, liposomes, resealed erythrocytes, and immunologically based systems may also be used to administer gene therapy vectors according to the methods of the invention.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In general, the composition should be sterile, and should be fluid so that easy syringability exists. Preferred pharmaceutical formulations are stable under the conditions of manufacture and storage and may be preserved against the contaminating action of microorganisms such as bacteria and fungi. In general, the relevant carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of injectable compositions can be brought about by including in the composition an agent which delays absorption, for example, aluminum monostearate and gelatin.

Sterile injectable solutions can be prepared by incorporating the active agents (vectors) in the required amount in an appropriate solvent with one or a combination of ingredients enumerated above, as required, followed by filtered sterilization. Preferably solutions for injection are free of endotoxin. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For topical applications, the pharmaceutical compositions may be formulated in a suitable ointment containing the pharmaceutically active component suspended or dissolved in one or more pharmaceutically acceptable carriers. Pharmaceutically acceptable carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For local delivery to the eye, the pharmaceutical compositions provided herein may be appropriately formulated, for example (but not limited to), in isotonic, pH adjusted sterile saline or water, either with or without a preservative such as benzylalkonium chloride. Alternatively, for ophthalmic uses, the pharmaceutical compositions may be formulated in an ointment such as petrolatum or as eye drops.

Methods of local administration to the eye include, e.g., choroidal injection, transscleral injection or placing a scleral patch, selective arterial catheterization, eye drops or eye ointments, intraocular administration including transretinal, subconjunctival bulbar, intravitreal injection, suprachoroidal injection, subtenon injection, scleral pocket and scleral cutdown injection, by osmotic pump, etc. The vectors or natural amino acid-containing compstatin peptides produced by other means can also be alternatively administered intravascularly, such as intravenously (IV) or intraarterially. In choroidal injection and scleral patching, the clinician or handler uses a local approach to the eye after initiation of appropriate anesthesia, including painkillers and ophthalmoplegics. A needle containing the pharmaceutical composition is directed into the subject's choroid or sclera and inserted under sterile conditions. When the needle is properly positioned, the composition is injected into either or both of the choroid or sclera. When using either of these methods, the clinician or handler can choose a sustained release or longer acting formulation. Thus, the procedure can be repeated only every several months or several years, depending on the subject's tolerance of the treatment and response.

Intraocular administration of drugs is well known in the art. See, e.g., U.S. Pat. Nos. 5,632,984 and 5,770,589 and U.S. Pub. No. 2016/0060297 A1. U.S. Pat. No. 6,378,526 provides methods for intrascleral injection of a therapeutic or diagnostic material at a location overlying the retina, which provide a minimally invasive technique for delivering the agent to the posterior segment of the eye.

In certain embodiments, a pharmaceutical composition containing vectors comprising the transgenes described herein is delivered to the vicinity of the eye, e.g., in close proximity to the posterior segment of the eye. The “vicinity of the eye” refers to locations within the orbit, which is the cavity within the skull in which the eye and its appendages are situated. Typically the compositions would be delivered close to their intended target within the eye, e.g., close to (within several millimeters of) the portion of the sclera that overlies the posterior segment of the eye, or immediately adjacent to the exterior surface of the sclera. In a preferred embodiment, the pharmaceutical compositions of the present invention are delivered into the vitreous cavity of the eye (i.e., intravitreally).

A number of polymeric delivery vehicles for providing controlled release have been used in an ocular context and can be used to administer the pharmaceutical compositions of the invention. Various polymers, e.g., biocompatible polymers, which may be biodegradable, can be used. For example, U.S. Pat. No. 6,692,759 describes methods for making an implantable device for providing controlled release of therapeutic agents in the eye. Other useful polymers and delivery systems for ocular administration of a therapeutic agent have been described. The active agent may be released as the polymer degrades. Polymers that have been used for drug delivery include, but are not limited to, poly(lactic-co-glycolic acid), polyanhydrides, ethylene vinyl acetate, polyglycolic acid, chitosan, polyorthoesters, polyethers, polylactic acid, and poly(beta amino esters). Peptides, proteins such as collagen and albumin, and dendrimers (e.g., PAMAM dendrimers) have also been used. Any of these can be used in various embodiments of the invention.

Poly (ortho esters) have been introduced into the eye and demonstrated favorable properties for sustained release ocular drug delivery (see Einmahl, S., 2002, Invest. Ophthalmol. Vis. Sci. 43 (5)). Polylactide particles have been used to target an agent to the retina and RPE following intravitreal injection of a suspension of such particles (Bourges et al., 2003, Invest. Ophthalmol. Vis. Sci. 44 (8)). A macroscopic implantable device suitable for introduction into the posterior or anterior segment of the eye is referred to herein as an ocular implant (see Jaffe, G., 2000, Invest. Ophthalmol. Hs. Sci., 41 (11)). Therefore, provided herein is an ocular implant comprising the vectors and/or compstatin analog polypeptides described herein and, optionally, other suitable transgenes, and other therapeutic agents to an individual. Such devices may be macroscopic implants comprising the pharmaceutical composition or may be comprised of a plurality of nanoparticles or microparticles impregnated with or encapsulating the agent. In one embodiment, the ocular implant is any ocular implant known in the art. Exemplary implants and methods for manufacture thereof are described, e.g., in US 2009/0220572 A1. Other implants known in the art can also be used.

Other embodiments include gel-forming compositions comprising a soluble collagen that are useful for the delivery of therapeutics to the posterior segment of the eye. The collagen is initially soluble and forms a solution that has a low viscosity but is capable of rapid formation of a gel under appropriate conditions, e.g., conditions encountered upon administration to a mammalian subject. The invention therefore provides a system for delivery of the pharmaceutically active agents to the posterior segment of the eye. The system is designed to localize such molecules in sufficient concentration to provide sustained delivery while at the same time allowing the macromolecule to be released in sufficient amounts. In addition, the collagen gel may protect the vectors and proteins produced therefrom from degradation.

The composition forms a gel following introduction into the body, e.g., upon contact with a physiological fluid. The composition can also form a gel upon contact with a fluid such as phosphate buffered saline, or other fluid containing appropriate ions. Thus the composition can be injected at an appropriate location, e.g., in close proximity to the posterior segment of the eye, where it forms a gel. Alternately, a preshaped gel implant can be made, e.g., by introducing the solution into a mold or cavity of the desired shape and allowing gel formation to occur in the presence of a suitable concentration of a salt. The salt can be added either prior to or following the introduction of the solution into the mold or cavity. The mold or cavity can be, e.g., any structure that contains a hollow space or concave depression into which a solution can be introduced. In another embodiment, a film or membrane is formed from the collagen solution containing a therapeutic agent.

For the treatment of chronic or acute lung conditions in which complement activation is implicated or which can benefit from other gene therapy, a preferred route of administration of a pharmaceutical composition is pulmonary administration. Accordingly, a pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers.

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, including replacement pulmonary surfactant, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intraarticular, intravitreal, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution can be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, in microbubbles for ultrasound-released delivery or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents including replacement pulmonary surfactants; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, PA.

### Uses and Therapeutic Administration of Compstatin Analog Transgenes and Natural Amino Acid-Containing Compstatins:

The compstatin analog transgenes or natural amino acid-containing peptides described herein are of practical utility for many purposes. In one embodiment, they can be used for any purpose for which compstatin and its analogs are utilized, if such use is amenable to production of the analogs from a transgene. Thus, in certain embodiments, the methods of regulating complement activation apply to living patients or subjects and comprise part or all of a method of treating the patient for a disease or disorder associated with complement activation, particularly alternative pathway (AP)-mediated complement activation, which can amplify complement effector responses and exacerbate inflammatory damage in tissues and cells, irrespective of the triggering mechanism of complement activation. Many such pathological conditions are known in the art and include, but are not limited to, atypical hemolytic uremic syndrome (aHUS); dense deposit disease (DDD); C3 glomerulonephritis (C3GN); C3 glomerulopathies; other complement-mediated nephropathies and glomerular inflammatory diseases; age-related macular degeneration (AMD); any eye disorder characterized by macular degeneration, choroidal neovascularization (CNV); retinal Neovascularization (RNV), proliferative vitreoretinopathy, glaucoma, uveitis, ocular inflammation, or any combination of these; paroxysmal nocturnal hemoglobinuria (PNH); cold agglutinin disease (CAD); warm antibody autoimmune hemolytic anemias (wAIHAs); sickle cell disease; transplant-associated thrombotic microangiopathies; rheumatoid arthritis (RA), systemic lupus erythematosus (SLE); several autoimmune and autoinflammatory kidney diseases; autoimmune myocarditis; multiple sclerosis; traumatic brain and spinal cord injury; cerebral, intestinal and renal ischemia-reperfusion (IR) injury; spontaneous and recurrent pregnancy loss; antiphospholipid syndrome (APS); Parkinson's disease; Alzheimer's disease; other neurodegenerative inflammatory conditions underpinned by aberrant synaptic remodeling, excessive microglial activity and cognitive decline; asthma; anti-nuclear cytoplasmic antigen-associated pauci-immune vasculitis (Wegener's syndrome); non-lupus autoimmune skin diseases such as pemphigus, bullous pemphigoid, and epidermolysis bullosa; post-traumatic shock, cancer; periodontitis; gingivitis; and atherosclerosis. In particular embodiments, the pathological condition has been associated with mutations and polymorphisms in the gene encoding FH and/or CD46, including but not limited to: AMD, aHUS and membrano-proliferative glomerulonephritis type II (MPGN-II, also referred to as dense deposit disease (DDD)). In other embodiments, the compstatin analogs produced by expression of polynucleotides as described herein are suitable for use as a substitute for Eculizumab or pegcetacoplan, in treatment of diseases for which those agents are currently prescribed, or for which they are being developed in pre-clinical and clinical studies. Those diseases include, but are not limited to, aHUS, PNH, C3G (DDD/C3GN), CAD, AMD, NMOSD (neuromyelitis optical spectrum disorder), generalized myasthenia gravis and amyotrophic lateral sclerosis (ALS).

### Blood-Related and Vessel-Related Disorders

In particular embodiments, a compstatin analog transgene is administered to a subject suffering from, or at risk of, a complement-mediated blood-related disorder, such as paroxysmal nocturnal hemoglobinuria (P H), atypical hemolytic uremic syndrome (aHUS), autoimmune hemolytic anemia, chronic cold agglutinin disease, HELLP syndrome, and/or warm autoimmune hemolytic anemia. In some embodiments, a compstatin analog transgene is administered to a subject suffering from, or at risk of, a complement-mediated disorder that affects the circulatory system. For example, in some embodiments, the disorder is thrombotic microangiopathy (TMA) or a vasculitis (e.g., IgA vasculitis) or other disorder associated with vessel inflammation, e.g., blood vessel and/or lymph vessel inflammation. In some embodiments, a vasculitis is polyarteritis nodosa, hypocomplementemic urticarial vasculitis, pulmonary vasculitis, Wegener's granulomatosis, giant cell arteritis, Churg-Strauss syndrome, microscopic polyangiitis, pauci-immune vasculitis, Henoch-Schonlein purpura, Takayasu's arteritis, Kawasaki disease, or Behcet's disease. In some embodiments, a disorder is TMA secondary to atypical hemolytic uremic syndrome. In some embodiments, a subject is positive for antineutrophil cytoplasmic antibody (ANCA).

### Eye Disorders

In some embodiments, a compstatin analog transgene is administered to a subject for treatment of a complement-mediated eye disorder, such as macular degeneration (e.g., age-related macular degeneration (AMD) or Stargardt macular dystrophy), diabetic retinopathy, glaucoma, or uveitis (e.g., posterior uveitis or anterior uveitis). In some embodiments, a subject suffers from or is at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease.

### Nervous System Disorders

In some embodiments, a compstatin analog transgene is used to treat a subject suffering from or at risk of a complement-mediated disorder that affects the nervous system, e.g., the central nervous system (CNS) and/or peripheral nervous system (PNS). Examples of such disorders include, e.g., a neurodegenerative disorder such as multiple sclerosis, other demyelinating diseases (e.g., neuromyelitis optica or chronic inflammatory demyelinating polyneuropathy (CIDP)), amyotrophic lateral sclerosis, chronic pain, fibromyalgia, stroke, intracerebral hemorrhage, allergic neuritis, acute or recurrent optic neuritis with myelin oligodendrocyte glycoprotein (MOG) or aquaporin (AQP)-4 antibodies, diabetic neuropathy, Huntington's disease, schizophrenia, Alzheimer's disease, Parkinson's disease, progressive supranuclear palsy, Lewy body dementia (i.e., dementia with Lewy bodies or Parkinson's disease dementia), frontotemporal dementia, progressive supranuclear palsy, corticobasal syndrome, Pick's disease, mild cognitive impairment, traumatic brain injury, traumatic spinal cord injury, multisystem atrophy, chronic traumatic encephalopathy, Creutzfeldt-Jakob disease, Guillain Barre Syndrome, glioblastoma, and leptomeningeal metastasis. In some embodiments, a subject suffers from neuropathic pain, e.g., arising from lesions that involve the somatosensory pathways with damage to small fibers in peripheral nerves and/or to the spino-thalamocortical system in the CNS.

### Kidney Disorders

In some embodiments, a compstatin analog transgene is used to treat a subject suffering from, or at risk of, a complement-mediated kidney disorder. Such disorders include, e.g., nephritis, e.g., glomerulonephritis, e.g., membranoproliferative glomerulonephritis (MPGN) (e.g., MPGN type I, MPGN type II, or MPGN type III), e.g., immune complex membranoproliferative glomerulonephritis (IC-MPGN). In some embodiments the disorder is IgA nephropathy (IgAN), primary membranous nephropathy, or diabetic nephropathy. In some embodiments, the disorder is polycystic kidney disease (PKD). In some embodiments, the disorder is C3 glomerulopathy. In some embodiments the disorder is characterized by glomerular deposits containing one or more complement activation products, e.g., C3b, in the kidney. In some embodiments treatment as described herein reduces the level of such deposits. In some embodiments a subject suffering from a complement-mediated kidney disorder suffers from proteinuria (an abnormally high level of protein in the urine) and/or an abnormally low glomerular filtration rate (GFR). In some embodiments treatment as described herein results in decreased proteinuria and/or an increased or stabilized GFR.

In these embodiments, the treatment methods typically comprise (1) identifying a subject with a disease or condition treatable by regulation of complement activation as described hereinabove (“complement mediated” disease, condition or disorder), (2) measuring a parameter of the disease or condition treatable by regulation of complement activation using art-standard techniques well within the purview of the skilled artisan (e.g., biopsy, histology, MRI, bone-scan, X-Ray, pain tolerance, posture, and the like), (3) administering to the subject an effective amount of a compstatin analog transgene of the invention using a treatment regimen and duration appropriate for the condition being treated, and (4) measuring the parameter of the disease or condition as an indication that the disease or condition has been ameliorated or has been treated. Delivery of the transgene may be performed by any suitable route of administration known in the art, as described herein. Development of appropriate dosages and treatment regimens will vary depending upon any number of factors, including but not limited to, the type of patient and type of disease state being treated, the age of the patient and the route of administration. The skilled artisan is familiar with the design of dosage regimens that take such variables into account.

In some embodiments a transgene encoding a compstatin analog may be administered systemically, e.g., intravenously or subcutaneously, for treatment of a complement-mediated disorder described herein. In some embodiments a local administration route may be used, e.g., where the disorder primarily affects a particular body system, organ, or tissue. For example, in some embodiments a transgene encoding a compstatin analog may be administered intraocularly (e.g., intravitreally) for treating an eye disorder. In some embodiments a transgene encoding a compstatin analog is administered by the pulmonary route (e.g., for treating a disorder affecting the respiratory system). In some embodiments, a transgene encoding a compstatin analog is administered intrathecally for treatment of a complement-mediated disorder that affects the central nervous system as described herein. In some embodiments intracisternal or intracerebroventricular administration may be used for treatment of a complement-mediated disorder that affects the central nervous system.

In another embodiment, compstatin analog transgenes are introduced into cells of a living host in conjunction with other gene therapy to improve efficacy of viral vector delivery and performance. Examples of such diseases or disorders include, but are not limited to, lysosomal storage diseases/disorders, such as Santavuori-Haltia disease (Infantile Neuronal Ceroid Lipofuscinosis Type 1), Jansky-Bielschowsky Disease (late infantile neuronal ceroid lipofuscinosis, Type 2), Batten disease Juvenile neuronal ceroid lipofuscinosis, Type 3), Kufs disease (neuronal ceroid lipofuscinosis, Type 4), Von Gierke disease (glycogen storage disease, Type Ia), glycogen storage disease, Type 1b, Pompe disease (glycogen storage disease, Type II), Forbes or Cori disease (glycogen storage disease, Type III), mucolipidosis II (I-Cell disease), mucolipidosis III (Pseudo-Hurler polydystrophy), mucolipdosis IV (sialolipidosis), cystinosis (adult nonnephropathic type), cystinosis (infantile nephropathic type), cystinosis Guvenile or adolescent nephropathic), Salla disease/infantile sialic acid storage disorder, and saposin deficiencies; disorders of lipid and sphingolipid degradation, such as GMI gangliosidosis (infantile, late infantile/juvenile, and adult/chronic), Tay-Sachs disease, Sandhoff disease, GM2 gangliodisosis, Ab variant, Fabry disease, Gaucher disease, Types I, II and III, metachromatic leukidystrophy, Krabbe disease (early and late onset), Neimann-Pick disease, Types A, B, C1, and C2, Farber disease, and Wolman disease (cholesteryl esther storage disease); disorders of mucopolysaccharide degradation, such as Hurler syndrome (MPSI), Scheie syndrome (MPS IS), Hurler-Scheie syndrome (MPS IH/S), Hunter syndrome (MPS II), Sanfillippo A syndrome (MPS IIIA), Sanfillippo B syndrome (MPS IIIB), Sanfillippo C syndrome (MPS IIIC), Sanfillippo D syndrome (MPS IIID), Morquio A syndrome (MPS IVA), Morquio B syndrome (MPS IVB), Maroteaux-Lamy syndrome (MPS VI), and Sly syndrome (MPS VII); disorders of glycoprotein degradation, such as alpha mannosidosis, beta mannosidosis, fucosidosis, asparylglucosaminuria, mucolipidosis I (sialidosis), galactosialidosis, Schindler disease, and Schindler disease, Type II/Kanzaki disease; and leukodystrophy diseases/disorders, such as abetalipoproteinemia, neonatal adrenoleukodystrophy, Canavan disease, cerebrotendinous xanthromatosis, Pelizaeus Merzbacher disease, Tangier disease, Refum disease, infantile, and Refum disease, classic.

Additional examples of such diseases/disorders of a subject as described herein include, but are not limited to, acid maltase deficiency (e.g., Pompe disease, glycogenosis type 2, lysosomal storage disease); carnitine deficiency; carnitine palmityl transferase deficiency; debrancher enzyme deficiency (e.g., Cori or Forbes disease, glycogenosis type 3); lactate dehydrogenase deficiency (e.g., glycogenosis type 11); myoadenylate deaminase deficiency; phosphofructokinase deficiency (e.g., Tarui disease, glycogenosis type 7); phosphogylcerate kinase deficiency (e.g., glycogenosis type 9); phosphogylcerate mutase deficiency (e.g., glycogenosis type 10); phosphorylase deficiency (e.g., McArdle disease, myophosphorylase deficiency, glycogenosis type 5); Gaucher's Disease (e.g., chromosome 1, enzyme glucocerebrosidase affected); Achondroplasia (e.g., chromosome 4, fibroblast growth factor receptor 3 affected); Huntington's Disease (e.g., chromosome 4, huntingtin); Hemochromatosis (e.g., chromosome 6, HFE protein); Cystic Fibrosis (e.g., chromosome 7, CFTR); Friedreich's Ataxia (chromosome 9, frataxin); Best Disease (chromosome 11, VMD2); Sickle Cell Disease (chromosome 11, hemoglobin); Phenylketoniuria (chromosome 12, phenylalanine hydroxylase); Marfan's Syndrome (chromosome 15, fibrillin); Myotonic Dystophy (chromosome 19, dystophia myotonica protein kinase); Adrenoleukodystrophy (x-chromosome, lignoceroyl-CoA ligase in peroxisomes); Duchene's Muscular Dystrophy (x-chromosome, dystrophin); Rett Syndrome (x-chromosome, methylCpG-binding protein 2); Leber's Hereditary Optic Neuropathy (mitochondria, respiratory proteins); Mitochondria Encephalopathy, Lactic Acidosis and Stroke (MELAS) (mitochondria, transfer RNA); and Enzyme deficiencies of the Urea Cycle.

Still additional examples of such diseases or disorders include, but are not limited to, Sickle Cell Anemia, Myotubular Myopathy, Hemophilia B, Lipoprotein lipase deficiency, Ornithine Transcarbamylase Deficiency, Crigler-Najjar Syndrome, Mucolipidosis IV, Niemann-Pick A, Sanfilippo A, Sanfilippo B, Sanfilippo C, Sanfilippo D, b-thalassaemia and Duchenne Muscular Dystrophy. Still further examples of diseases or disorders include those that are the result of defects in lipid and sphingolipid degradation, mucopolysaccharide degradation, glycoprotein degradation, leukodystrophies, and the like.

Improvement in viral vector-mediated gene therapy efficacy by combined administration with the compstatin analog transgenes of the invention can be seen in many ways. For instance, in some embodiments, the combined administration allows administration of the gene therapy at a reduced dose, at a reduced number of doses, and/or at a reduced frequency of dosage compared to an effective dosing regimen for the gene therapy alone, and these differences are easily measured. Efficacy of gene therapy in this regard can be is assessed at, e.g., 1 week, 2 weeks, 3 weeks, 4 weeks, 6 weeks, 8 weeks, 3 months, 6 months, or longer after administration of therapy.

In some embodiments, efficacy of gene therapy is measured or indicated by a disease sign or symptom recurrence-free period.

In some embodiments, a humoral response and/or a cellular response to administration of viral vectors can be measured. For example, decrease in humoral response is measured or indicated by decrease in magnitude of response or fold decrease from baseline of antibody (e.g., neutralizing antibody) levels. In some embodiments, antibody level is level of antibody against viral vector, e.g., capsid protein. In some embodiments, baseline is a value, level, amount or quantity measured or indicated in a subject prior to administration of gene therapy, or a control subject not receiving the gene therapy. In some embodiments, decreased humoral response is indicated by a decrease in antibody titer from baseline of at least 10%, 20%, 30%, 40%, 50%, 60%, 70%, 80%, 90%, or 100%.

Cellular response is indicated or measured by secretion of granzyme B (GrB) and/or IFNy. In some embodiments, decrease in cellular response is measured or indicated by decrease in magnitude of response or fold decrease from baseline of GrB and/or IFNy levels. In some embodiments, decreased cellular response is indicated by a decrease in GrB and/or IFNy levels from baseline of at least 10%, 20%, 30%, 40%, 50%, 60%, 70%, 80%, 90%, or 100%. In some embodiments, baseline is a value, level, amount or quantity measured or indicated in a subject prior to administration of gene therapy, or in a control subject not administered the gene therapy.

In some embodiments, efficacy of gene therapy is measured by level of presence or expression of a compstatin analog transgene or other transgene described herein, and/or level or activity of a protein encoded by the transgene(s). For example, combined delivery of a compstatin analog transgene and other gene therapy results in a level of transgene in a subject (e.g., in a cell or tissue of the subject) at, e.g., 1 week, 2 weeks, 3 weeks, 4 weeks, 6 weeks, 8 weeks, 3 months, 6 months, or longer after combined therapy, that is at least 10%, 20%, 30%, 40%, 50%, 60%, 70%, 80%, 90%, 100%, 125%, 150%, 175%, 200%, or more, higher relative to a corresponding level of transgene in a subject not administered the compstatin analog transgene.

In some embodiments, the efficacy of gene therapy is measured by a stable level of expression of the transgene in the subject over a period of, e.g., I week, 2 weeks, 3 weeks, 4 weeks, 6 weeks, 8 weeks, 3 months, 6 months, or longer, relative to a corresponding level of expression of the transgene over the same period in a control subject (e.g., a control subject receiving the gene therapy and not administered the compstatin analog transgene). In some embodiments, a stable level of expression is a level of expression that differs by no more than 30%, 25%, 20%, 15%, 10%, or 5% over a defined period of time.

In some embodiments, efficacy of gene therapy with a transgene that encodes an inhibitor of a target gene or polypeptide is measured by level of expression of a target gene and/or level of expression and/or activity of a target polypeptide. In some embodiments, combined therapy of a compstatin analog transgene and other gene therapy results in a level of expression of a target gene and/or level of expression and/or activity of a target polypeptide in a subject (e.g., in a cell or tissue of the subject) at, e.g., 1 week, 2 weeks, 3 weeks, 4 weeks, 6 weeks, 8 weeks, 3 months, 6 months, or longer after combined therapy, that is at least 10%, 20%, 30%, 40%, 50%, 60%, 70%, 80%, 90%, or 100% lower relative to a corresponding level of expression and/or activity in a subject not administered the compstatin analog transgene.

In another embodiment, compstatin analog transgenes are used together with other transgenes in a combination treatment of diseases or conditions in which complement activation and other gene therapy-treatable dysfunctions both contribute to the disease or condition.

In some embodiments, the therapeutic effect of combined administration of a compstatin analog transgene and the additional gene therapy for the disease or condition, or a symptom thereof, is greater than the effect produced by either the gene therapy or treatment with the compstatin analog alone. The difference between the combined effect and the effect of the gene therapy alone or the compstatin analogy alone can be a statistically significant difference. In some embodiments, the combined result is synergistic.

These diseases or conditions can be any one, or combination, of the diseases and conditions listed herein, in which complement activation plays a role and which may benefit from one or more of the gene therapies listed above.

As a specific example, the vascular endothelial growth factor (VEGF) family of growth factors controls pathological angiogenesis and increased vascular permeability in important eye diseases such as diabetic retinopathy (DR) and age-related macular degeneration (AMD). Complement activation also plays an important role in the pathogenesis of these diseases. As such, vectors encoding compstatin analog transgenes combined with transgenes for the control of VEGF can provide a dual-mechanism therapy.

In certain embodiments, VEGF-controlling transgenes, such as those mentioned elsewhere herein, together with the compstatin analog transgenes of the invention, are administered via viral vectors as described herein. The compstatin analogs produced by expression of the transgene in target cells and tissues serve a dual function: (1) they reduce immunogenicity of the viral vectors, thus improving survival of the vectors and production of the VEGF-controlling gene products; and (2) they directly treat the underlying disease or condition.

The VEGF-controlling transgenes and the compstatin analog transgenes can be incorporated into one, or more than one, viral vector. In one embodiment, both transgenes are included on a single viral vector. In another embodiment, the compstatin analog transgene is arranged with the VEGF-controlling transgene to produce a fusion protein, wherein the compstatin analog is linked to the VEGF-controlling gene product. The linkage may be direct, or there may be a spacer or linker between the compstatin analog and the VEGF-controlling gene product.

In some embodiments, transgenes encoding a compstatin analog as described herein and a VEGF inhibitor may be used to treat a subject suffering from or at risk of a complement-mediated disorder that is characterized by pathologic angiogenesis and/or pathologic blood vessel permeability or is associated with increased risk of developing pathologic angiogenesis and/or pathologic blood vessel permeability. In some embodiments the protein is a modified VEGF-inhibiting Fab having a compstatin analog fused to a heavy chain or a light chain of the Fab, as described herein. Pathologic angiogenesis and/or pathologic blood vessel permeability refers to angiogenesis or blood vessel permeability that is aberrant, excessive, and/or otherwise detrimental to a subject who experiences it. In some embodiments the disorder is an eye disorder. In some embodiments the eye disorder is AMD. In some embodiments the protein is administered to an eye suffering from GA, wherein the eye has not been diagnosed with neovascular AMD. In some embodiments the protein is administered to an eye suffering from neovascular AMD, wherein the eye has not been diagnosed with GA. In some embodiments the protein is administered to an eye suffering from neovascular AMD and GA. In some embodiments the protein is administered to an eye suffering from neovascular AMD and intermediate AMD. In some embodiments the protein is administered to an eye suffering from neovascular AMD, wherein the eye has not been diagnosed with GA. In some embodiments the eye disorder is choroidal neovascularization (CNV), which may be a manifestation of AMD or may result from other causes. In some embodiments the eye disorder is proliferative diabetic retinopathy, neovascular glaucoma, diabetic macular edema, retinopathy of prematurity, or macular edema secondary to retinal vein occlusions. In some embodiments the disorder is cancer.

In yet another embodiment, compstatin analog peptides themselves are used instead of their transgenes to improve the efficacy of gene therapy and/or to provide a synergistic effect with the gene therapy. These methods involve administering one or more compstatin analog peptides to a subject receiving, or who has previously received, or will be receiving, gene therapy as described herein.

In certain embodiments, the compstatin analog is administered to a subject who has received or is concurrently or sequentially receiving one or more doses of gene therapy as discussed herein. In some embodiments, a subject has received gene therapy 1 day, 1 week, 2 weeks, 4 weeks, 2 months, 4 months, 6 months, or more prior to administration of the compstatin analog. In other embodiments, a compstatin analog is administered to a subject who has not been receiving gene therapy, but is likely to receive gene therapy in the future.

In some embodiments, the compstatin analog and the gene therapy are administered concurrently (e.g., within about 1 minute, 5 minutes, 10 minutes, 15 minutes, 30 minutes, 45 minutes, 1 hour, or 2 hours of each other). In some embodiments, the compstatin analog and the gene therapy are administered sequentially (e.g., more than 1 hour, 6 hours, 12 hours, 18 hours, 24 hours, 2 days, 3 days, 4 days, 5 days, 6 days, 7 days, 2 weeks, 4 weeks, or more, apart). In some embodiments, a subject is pretreated with a compstatin analog before receiving a gene therapy, e.g., a few minutes to 2, 4, 6, 8, 10, 12, 14, 16, 18, 20, or 24 hours or more before a gene therapy dose.

Pretreatment, concurrent treatment, or post-treatment may comprise a single dose or multiple doses of compstatin analog, e.g., 2, 3, 4, 5, 6, 7, 8, 9, or 10 doses. If multiple doses are administered, they may be administered over the course of several minutes, or hours, or days before, during or after the gene therapy. Administration of compstatin analog may be interspersed with multiple courses of the gene therapy.

In some embodiments, a compstatin analog is administered for a period of time for the purpose of inhibiting complement activation until the viral vector of the gene therapy is taken up by one or more target cells (e.g., taken up by a target cell by a certain level or extent). Cell uptake is indicated or measured in a subject using an assay to detect a decreased level of viral vector in a sample obtained from the subject (e.g., a serum sample) and/or increased level of viral vector in one or more target cells. In some embodiments, a compstatin analog is administered in conjunction with immunosuppressive therapy.

The embodiments that utilize pre-formed compstatin analogs (instead of or in addition to the transgene), can involve delivery methods that include a combination of administration routes and doses. For instance, compositions comprising the peptide can be administered orally, subcutaneously, intravenously, ophthalmically or intramuscularly and the vectors comprising transgenes can be administered by the same route or a different route, at the same time or at a different time, as described above.

In some embodiments, the combination therapy results in improvement of the gene therapy (e.g., an improvement in a disease or disorder described herein or a symptom thereof) in a subject over a specified time period, as discussed above with embodiments involving the use of a compstatin analog transgene.

The following example is provided to describe the invention in greater detail. It is intended to illustrate, no to limit, the invention.

### Example 1. Efficacy of Compstatin Analog Cp50 on Laser-Induced Choroidal Neovascularization in a Non-Human Primate Model of Wet-Type Age-Related Macular Degeneration

The effects of the compstatin analogue Cp50 (described above) were compared vs. vehicle, in a nonhuman primate model of wet-type age-related macular degeneration (AMD). In this model, laser-induced choroidal neovascularization (CNV) was performed in the eyes of macaques.

### Study Design

Laser-induced CNV was performed in both eyes of the macaques, as described below. Two weeks (Day 14) after laser photocoagulation, laser lesions were graded (Grade 1-4) via fluorescein angiograms (FA). Eyes that displayed a high ratio (percentage) of Grade 4 lesions were selected and randomized into treatment groups.

Under ketamine (15 mg/kg, i.m.) and xylazine (2 mg/kg, i.m), on Day 15, the eyes were intravitreally (IVT) injected with either vehicle, or compstatin Cp50. FA measurements were performed on weeks 4, 6, 8 10 and 12 (total of five times), following CNV.

### Methods

### Laser-Induced Choroidal Neovascularization (CNV)

Briefly, under light ketamine (10 mg/kg, i.m.) anesthesia, the pupils were dilated with tropicamide/phenylephrine (Mydrin-P® ophthalmic solution; Santen Pharmaceutical Co., Osaka, Japan). For the laser treatment, the macaques were anesthetized with a combination of ketamine (15 mg/kg, i.m.; Daiichi Sankyo Propharma Co., Tokyo, Japan) and xylazine (2 mg/kg, i.m.; Bayer Yakuhin, Osaka, Japan). A green laser (900 mW output; Green Scan Laser Photocoagulator, GYC-500, Nidek, Co., Gamagori, Japan) was applied to eight spots (about 75 μm, 0.1 s duration) around the macula in a grid pattern. The extent of CNV lesion was assessed using fluorescence angiography (FA) at predetermined time points.

On the day of lesion grading, under light ketamine (10 mg/kg, i.m.) anesthesia, the pupils were dilated with tropicamide/phenylephrine (Mydrin-P® ophthalmic solution), and then the macaques were anesthetized with a combination of ketamine (15 mg/kg, i.m.) and xylazine (2 mg/kg, i.m).

### Fluorescence Angiography (FA)

Fluorescence Angiography (FA) was used to check the extent of CNV in the back of the eye. Ten percent sodium fluorescein (Fluorescite®; 10 mg/kg, i.v.; Novartis, Tokyo, Japan) was injected and a fundus camera took pictures of the back of the eye at about 1, 3, and 5 min after i.v. injection. Grading of the lesions was as follows:
Grade 1: no hyperfluorescence.
Grade 2: lesions exhibit hyperfluorescence without leakage.
Grade 3: lesions show hyperfluorescence in the early or mid-transit images and late leakage.
Grade 4: lesions show bright hyperfluorescence in the transit and late leakage beyond the treated areas.

Following measurement of FA, vitreous fluid was removed.

### Vitreous Injection

Following randomization, under ketamine (15 mg/kg, i.m.) and xylazine (2 mg/kg, i.m), on Day 15, the eyes were intravitreally (IVT) injected with 50 μl of either vehicle, or Cp50. Before IVT injection, eyes were treated twice daily for three days with levofloxacin hydrate (0.5%; Cravit®, Santen Pharmaceutical Co.).

Under anesthesia, a solution of tropicamide/phenylephrine HCl (Mydrin®-P), levofloxacin hydrate (0.5%; Santen Pharmaceutical Co.), and oxybuprocaine HCl (0.4%; Santen Pharmaceutical Co.) was instilled before dosing.

### Results

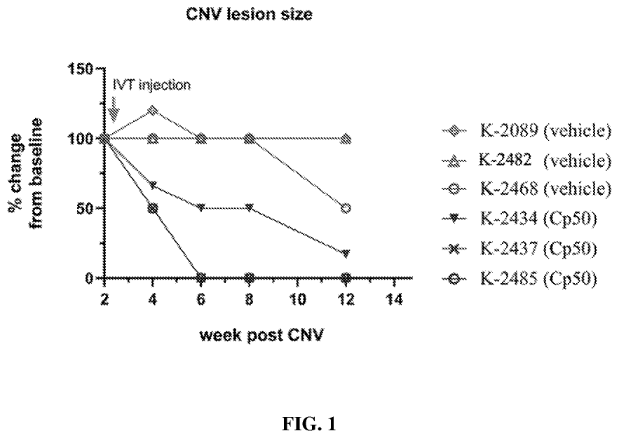

At Weeks 4, 6, 8 and 12, mean Grade 4 lesion of eyes treated with compstatin Cp50 were reduced compared to those of eyes treated with vehicle (FIG. 1). Two Cp50-treated eyes displayed a decline in CNV lesion size, evident as early as week 4 post laser-induced CNV. Treatment with Cp50 led to a complete (100%) reduction of Grade 4 lesion ratios from week 6 onwards and until the end of the experimental window of CNV lesion monitoring (at week 12). A third Cp50-treated animal (K-2434) displayed a consistent trend of reduction of CNV lesion size, dropping to 50% of baseline by week 6 and continuing to drop down to 25% by week 12. On the contrary, vehicle-treated eyes retained the Grade-4 score of CNV lesioning.

The present invention is not limited to the embodiments described and exemplified herein, but is capable of variation and modification within the scope of the appended claims.

What is claimed:

1. A compstatin analog comprising a peptide having an amino acid sequence
(SEQ ID NO: 4)
Xaal-[Cys-Ile-Trp-Gln-Asp-Trp-Gly-Xaa2-His-Arg-
Cys]-Xaa3-Xaa4;
wherein Xaa1 is absent or comprises the dipeptide Tyr-Ile, Xaa2 is Ala or Glu, Xaa3 is absent or is Ile, and Xaa4 is absent or represents one, two or three Lys residues, and wherein the Cys residues form a disulfide bond, to form a cyclic peptide comprising the sequence within the brackets.

2. The compstatin analog of claim 1, having an amino acid sequence
(SEQ ID NO: 5)
Tyr-Ile-[Cys-Ile-Trp-Gln-Asp-Trp-Gly-Xaa2-His-Arg-
Cys]-Ile-Xaa4;
wherein Xaa2 is Ala or Glu and Xaa4 represents two or three Lys residues.

3. The compstatin analog of claim 1 or claim 2, comprising
(SEQ ID NO: 6)
Tyr-Ile-[Cys-Ile-Trp-Gln-Asp-Trp-Gly-Ala-His-Arg-
Cys]-Ile-Lys-Lys-Lys
or
(SEQ ID NO: 7)
Tyr-Ile-[Cys-Ile-Trp-Gln-Asp-Trp-Gly-Glu-His-Arg-
Cys]-Ile-Lys-Lys-Lys

4. A peptide consisting essentially of SEQ ID NO:6 or SEQ ID NO:7.

5. A peptide consisting of SEQ ID NO:6 or SEQ ID NO:7.

6. The compstatin analog of claim 1, selected from the group consisting of SEQ ID NO:8, SEQ ID NO:9, SEQ ID NO: 10, SEQ ID NO:11, SEQ ID NO:12, SEQ ID NO: 13, SEQ ID NO: 14, SEQ ID NO:15, and SEQ ID NO:16.

7. A polynucleotide comprising a sequence that encodes the compstatin analog or peptide of any one of claims 1-6.

8. The polynucleotide of claim 7, which encodes SEQ ID NO:6 or SEQ ID NO:7.

9. The polynucleotide of claim 8, selected from SEQ ID NO:17 and SEQ ID NO:18.

10. The polynucleotide of claim 7, disposed within an expression cassette or a vector.

11. The polynucleotide of claim 10, wherein the vector is an expression vector.

12. The polynucleotide of claim 11, wherein the vector is adapted for expression in a procaryotic or eucaryotic expression system, and/or is used in gene therapy.

13. The polynucleotide of claim 12, wherein the vector is used in gene therapy and is selected from retrovirus, adenovirus, adeno-associated virus (AAV), and herpes simplex virus-1.

14. The polynucleotide of claim 13, wherein the vector is an adeno-associated viral (AAV) vector.

15. The polynucleotide of claim 14, wherein the AAV vector is an AAV1, AAV2, AAV3, AAV4, AAV5, AAV6, AAV7, AAV8, AAV9, AAV10, or AAV11 vector, or any variant thereof.

16. A vector comprising a compstatin analog polynucleotide that encodes a compstatin analog comprising SEQ ID NO:4, SEQ ID NO:5, SEQ ID NO:6 or SEQ ID NO:7.

17. The vector of claim 16, comprising at least one insertion site for at least one transgene for delivery of gene therapy.

18. The vector of claim 16, wherein the compstatin analog polynucleotide has a sequence of SEQ ID NO:17 or SEQ ID NO:18.

19. The vector of claim 16, selected from retrovirus, adenovirus, adeno-associated virus (AAV), and herpes simplex virus-1.

20. The vector of claim 19, which is an adeno-associated viral (AAV) vector.

21. The vector of claim 20, which is an AAV1, AAV2, AAV3, AAV4, AAV5, AAV6, AAV7, AAV8, AAV9, AAV10, or AAV11 vector, or any variant or combination thereof.

22. The vector of claim 16, comprising at least one other transgene for gene therapy.

23. The vector of claim 22, wherein the transgene encodes a therapeutic protein, enzyme, hormone, blood coagulation factor, cytokine, or growth factor.

24. The vector of claim 22, wherein the gene therapy is for the treatment of a blood disorder, an eye disorder, autoimmune disease, a muscle disorder, a neurological disorder, or cancer.

25. The vector of any one of claims 16 to 24, wherein the compstatin analog polynucleotide and/or transgene are adapted for tissue or organ-specific expression.

26. The vector of claim 22, wherein the compstatin analog polynucleotide and the transgene are arranged on the vector to produce a fusion protein comprising the compstatin analog and the transgene product.

27. The vector of claim 26, wherein the fusion protein comprises the compstatin analog directly linked to the transgene product.

28. The vector of claim 26, wherein the fusion protein comprises the compstatin analog linked to the transgene product via a linker or spacer.

29. The vector of claim 26, wherein the transgene encodes a VEGF inhibitor.

30. The vector of claim 29, wherein the VEGF inhibitor comprises an extracellular domain of a VEGF receptor.

31. The vector of claim 29, wherein the VEGF inhibitor comprises an antibody fragment selected from the group consisting of a Fab, F(ab′)2, Fv, scFv, or a single domain antibody.

32. The vector of claim 31, wherein the antibody fragment is a Fab comprising a VL domain, a CL domain, a VH domain, and a CH1 domain.

33. The vector of claim 32, encoding a fusion protein comprising the compstatin analog fused directly or indirectly to one, two, three, or four of:
(i) the VL of the Fab;
(ii) the CL of the Fab;
(iii) the VH of the Fab; or
(iv) the CHI of the Fab.

34. The vector of claim 33, encoding a fusion protein comprising the compstatin analog fused directly or indirectly to the N-terminus of the VL of the Fab, the C-terminus of the CL of the Fab, the N-terminus of the VH of the Fab, or the C-terminus of CHI of the Fab.

35. A pharmaceutical composition comprising at least one vector as claimed in any one of claims 16-34, and a pharmaceutically acceptable carrier.

36. The pharmaceutical composition of claim 35, formulated for administration by a route selected from subcutaneous, intradermal, intravenous, intraocular (including intravitreal and subretinal), intracerebral, intraperitoneal, intramuscular injection, periodontal administration (including gingival administration or intrapapillary infiltration injection), intranasal, epidural, oral, sublingual, intrathecal, intravaginal, transdermal, rectal, by inhalation, or topical.

37. The pharmaceutical composition of claim 35, formulated for systemic administration.

38. The pharmaceutical composition of claim 35, formulated for local administration.

39. The pharmaceutical composition of claim 38, wherein the local administration is to the brain and/or central nervous system, eye, lungs and/or respiratory system, heart and/or vascular system, lymphatic system, kidney, spleen, pancreas, liver, gastrointestinal system, periodontal tissue, skin, bone, joint or synovial fluid, or any combination thereof.

40. The pharmaceutical composition of claim 35, further comprising a compstatin analog having a sequence selected from SEQ ID NO:4, SEQ ID NO:5, SEQ ID NO:6 and SEQ ID NO: 7.

41. A pharmaceutical composition comprising a compstatin analog having a sequence selected from SEQ ID NO:4, SEQ ID NO:5, SEQ ID NO:6 and SEQ ID NO:7 in a pharmaceutically acceptable carrier.

42. The pharmaceutical composition of claim 41, formulated for administration by a route selected from subcutaneous, intradermal, intravenous, intraocular (including intravitreal, subretinal), intracerebral, intraperitoneal, intramuscular injection, periodontal administration (including gingival administration or intrapapillary infiltration injection), intranasal, epidural, oral, sublingual, intrathecal, intravaginal, transdermal, rectal, by inhalation, or topical.

43. The pharmaceutical composition of claim 41, formulated for systemic administration.

44. The pharmaceutical composition of claim 41, formulated for local administration.

45. The pharmaceutical composition of claim 44, wherein the local administration is to the brain and/or central nervous system, eye, lungs and/or respiratory system, heart and/or vascular system, lymphatic system, kidney, spleen, pancreas, liver, gastrointestinal system, periodontal tissue, skin, bone, joint or synovial fluid, or any combination thereof.

46. The pharmaceutical composition of claim 41, further comprising at least one vector as claimed in any one of claims 16-34.

47. A kit comprising a plurality of pharmaceutical compositions, wherein at least one of the pharmaceutical compositions is a peptide-containing composition comprising a compstatin analog having a sequence selected from SEQ ID NO:4, SEQ ID NO:5, SEQ ID NO:6 and SEQ ID NO: 7 in a pharmaceutically acceptable carrier, and at least one of the pharmaceutical compositions is a vector-containing composition comprising a vector as claimed in any one of claims 16-34, in a pharmaceutically acceptable carrier.

48. The kit of claim 47, wherein at least one of the peptide-containing composition and the vector-containing composition is formulated for systemic administration.

49. The kit of claim 47, wherein at least one of the peptide-containing composition and the vector-containing composition is formulated for local administration.

50. The kit of claim 47, wherein the peptide-containing composition is formulated for a selected route of administration and the vector-containing composition is formulated for a route of administration that is different than the route of administration selected for the peptide-containing composition, optionally comprising instructions for administering the peptide-containing composition on a schedule that is different from the schedule for administering the vector-containing composition.

51. A kit comprising a plurality of pharmaceutical compositions each of which is a vector-containing composition comprising a vector as claimed in any one of claims 16-34, in a pharmaceutically acceptable carrier.

52. The kit of claim 51, wherein at least one of the pharmaceutical compositions is formulated for systemic administration.

53. The kit of claim 51, wherein at least one of the pharmaceutical compositions is formulated for local administration.

54. The kit of claim 51, the pharmaceutical compositions are formulated for different routes of administration, optionally comprising instructions for administering one composition on a schedule that is different from the schedule for administering another composition.

55. A method of treating a subject having or at risk of a complement-mediated disorder, the method comprising administering to the subject a composition that includes at least one vector comprising a compstatin analog polynucleotide that encodes a compstatin analog of SEQ ID NO: 4, SEQ ID NO:5, SEQ ID NO:6 or SEQ ID NO:7.

56. The method of claim 55, wherein, after the administration of the composition, a level of complement activity in the subject or in a biological sample from the subject is reduced relative to a level before the administration of the composition, or in an equivalent subject who has not been administered the composition.

57. The method of claim 56, wherein the level of complement activity is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration or in the equivalent subject who has not been administered the composition.

58. The method of claim 55, wherein the composition is administered systemically to the subject.

59. The method of claim 55, wherein the composition is administered locally to tissues or organs of the subject.

60. The method of claim 55, wherein the vector is an adeno-associated viral (AAV) vector.

61. The method of claim 60, wherein the AAV vector is an AAV1, AAV2, AAV3 (e.g., AAV3B), AAV4, AAV5, AAV6, AAV7, AAV8, AAV9, AAV10, or AAV11 vector.

62. The method of claim 55, wherein the complement-mediated disorder is a chronic disorder.

63. The method of claim 55, wherein the complement-mediated disorder involves complement-mediated damage to red blood cells, optionally wherein the disorder is paroxysmal nocturnal hemoglobinuria or atypical hemolytic uremic syndrome.

64. The method of claim 55, wherein the complement-mediated disorder is an autoimmune disease, optionally wherein the disorder is multiple sclerosis.

65. The method of claim 55, wherein the complement-mediated disorder involves the kidney, optionally wherein the disorder is membranoproliferative glomerulonephritis, lupus nephritis, IgA nephropathy (IgAN), primary membranous nephropathy (primary MN), C3 glomerulopathy (C3G), or acute kidney injury.

66. The method of claim 55, wherein the complement-mediated disorder involves the central or peripheral nervous system or neuromuscular junction, optionally wherein the disorder is neuromyelitis optica, Guillain-Barre syndrome, amyotrophic lateral sclerosis, multifocal motor neuropathy, or myasthenia gravis.

67. The method of claim 55, wherein the complement-mediated disorder involves the respiratory system, optionally wherein the disorder is characterized by pulmonary fibrosis.

68. The method of claim 55, wherein the complement-mediated disorder involves the vascular system, optionally wherein the disorder is characterized by vasculitis.

69. The method of claim 55, where the composition is administered to the eye of a subject suffering from an eye disorder.

70. The method of claim 69, wherein the composition is administered intravitreally.

71. The method of claim 69, wherein the eye disorder is age-related macular degeneration (AMD).

72. The method of claim 71, wherein the eye has one or more of (i) geographic atrophy, (ii) wet AMD (riii) geographic atrophy and wet AMD, or (iv) intermediate AMD.

73. The method of claim 55, comprising the steps of:
(1) providing the subject;
(2) administering the composition to the subject, whereby the compstatin analog is produced in the subject; and
(3) measuring one or more parameters of the complement-mediated disorder.

74. The method of claim 73, wherein the measuring is performed before, during and/or after administration of the composition.

75. The method of claim 73, wherein the measuring is performed on an equivalent subject who is not administered the composition.

76. The method of claim 55, wherein the complement-mediated disorder is selected from the group consisting of atypical hemolytic uremic syndrome (aHUS); dense deposit disease (DDD); C3 glomerulonephritis (C3GN); C3 glomerulopathies; complement-mediated nephropathies and glomerular inflammatory diseases; age-related macular degeneration (AMD); eye disorder characterized by macular degeneration, choroidal neovascularization (CNV), retinal Neovascularization (RNV), proliferative vitreoretinopathy, glaucoma, uveitis, ocular inflammation, or any combination of these; paroxysmal nocturnal hemoglobinuria (PNH); cold agglutinin disease (CAD); warm antibody autoimmune hemolytic anemias (wAIHAs); sickle cell disease; transplant-associated thrombotic microangiopathies; rheumatoid arthritis (RA); systemic lupus erythematosus (SLE); autoimmune and autoinflammatory kidney diseases; autoimmune myocarditis; multiple sclerosis; traumatic brain and spinal cord injury; cerebral, intestinal and renal ischemia-reperfusion (IR) injury; spontaneous and recurrent pregnancy loss; anti-phospholipid syndrome (APS); Parkinson's disease; Alzheimer's disease; neurodegenerative inflammatory conditions underpinned by aberrant synaptic remodeling, microglial activity and cognitive decline; asthma; anti-nuclear cytoplasmic antigen-associated pauci-immune vasculitis (Wegener's syndrome); non-lupus autoimmune skin diseases such as pemphigus, bullous pemphigoid, and epidermolysis bullosa; post-traumatic shock; cancer; periodontitis; gingivitis; and atherosclerosis.

77. The method of any one of claims 55-76, wherein the subject is a non-human primate or a human.

78. The method of any one of claims 55-77, comprising administering more than one dose of the composition to the subject.

79. The method of claim 78, comprising administering doses of the composition to the subject at pre-determined time intervals.

80. The method of claim 79, wherein the time intervals are within hours, days, weeks or months of one another.

81. A method of treating a subject having or at risk of a disease or disorder having a complement-mediated component and one or more other component, the method comprising administering to the subject a composition that includes at least one vector comprising:
a) a compstatin analog polynucleotide that encodes a compstatin analog of SEQ ID NO:4, SEQ ID NO:5, SEQ ID NO:6 or SEQ ID NO:7 and
b) at least one transgene that encodes a gene product that treats the one or more other components.

82. The method of claim 81, wherein after the administration of the composition, a level of complement activation and/or the other component in the subject or in a biological sample from the subject is reduced or ameliorated relative to a level before the administration of the composition, or in an equivalent subject who has not been administered the composition.

83. The method of claim 82, wherein the level of complement activation and/or the other component is reduced or ameliorated by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration or in the equivalent subject who has not been administered the composition.

83. The method of claim 81, wherein the other component is associated with a neovascular phenotype.

84. The method of claim 81, wherein the subject is a non-human primate or a human.

85. The method of claim 81, where the composition is administered to the eye of a subject suffering from an eye disorder.

86. The method of claim 85, wherein the eye disorder is age-related macular degeneration (AMD).

87. The method of claim 86, wherein the transgene encodes a VEGF inhibitor.

88. The method of claim 81, wherein the vector is an adeno-associated viral (AAV) vector.

89. The method of claim 88, wherein the AAV vector is an AAV1, AAV2, AAV3 (e.g., AAV3B), AAV4, AAV5, AAV6, AAV7, AAV8, AAV9, AAV10, or AAV11 vector.

90. The method of claim 81, wherein the transgene encodes a therapeutic protein, enzyme, hormone, blood coagulation factor, cytokine, or growth factor.

91. The method of claim 81, wherein the compstatin analog polynucleotide and the transgene are arranged on a single vector to produce a fusion protein comprising the compstatin analog and the transgene product.

92. The method of claim 91, wherein the fusion protein comprises the compstatin analog linked to the transgene product directly or via a linker or spacer.

93. The method of claim 91, wherein the transgene encodes a VEGF inhibitor.

94. The method of claim 81, wherein the compstatin analog polypeptide and the at least one other transgene are disposed on two or more separate vectors.

95. The method of claim 94, wherein the vector comprising the compstatin analog and the vector or vectors comprising the at least one other transgene are administered by different routes.

96. The method of claim 95, wherein the vector comprising the compstatin analog is administered systemically and the vector or vectors comprising the at least one other transgene are administered locally.

97. The method of claim 94, wherein the vector comprising the compstatin analog is administered before, during or after administration of the vector or vectors comprising the at least one other transgene.

98. The method of claim 81, comprising the steps of:
(1) providing the subject;
(2) administering the composition to the subject, whereby the compstatin analog and the transgene are produced in the subject; and
(3) measuring one or more parameters of the complement-mediated disorder or other component.

99. The method of claim 98, wherein the measuring is performed before, during and/or after administration of the composition, and/or on an equivalent subject who is not administered the composition.

100. The method of any one of claims 81-99, comprising replacing the compstatin analog polynucleotide or the vector comprising the compstatin analog polynucleotide with the compstatin analog peptide itself.

101. A method of improving efficacy of gene therapy in a subject who will receive, is receiving or has received gene therapy, comprising administering to the subject a vector comprising a compstatin analog polynucleotide that produced in the subject a compstatin analog having SEQ ID NO:4, SEQ ID NO:5, SEQ ID NO:6 or SEQ ID NO:7, thereby improving the efficacy of the gene therapy.

102. The method of claim 101, wherein the efficacy of the gene therapy is improved in the subject over a selected time period.

103. The method of claim 102, wherein the selected time period is at least about 1 week, 2 weeks, 4 weeks, 2 months, 3 months, 6 months, or 1 year.

104. The method of claim 101, wherein the efficacy is assessed by observing or measuring a decrease in an immune response against the gene therapy.

105. The method of claim 101, wherein the efficacy is assessed by observing or measuring improvement in transduction of a viral vector carrying a transgene.

106. The method of claim 101, wherein the efficacy is assessed by observing or measuring a decrease in complement-mediated clearance of a viral vector carrying a transgene.

107. The method of any one of claims 101-106, wherein the efficacy is measured by (i) comparing a subject receiving the vector comprising the compstatin analog polynucleotide with a control subject not receiving the vector comprising the compstatin analog polynucleotide, and/or (ii) comparing a subject during or after receiving the vector comprising the compstatin analog polynucleotide with the same subject before receiving the vector comprising the compstatin analog polynucleotide.

108. The method of claim 101, wherein the vector comprising the compstatin analog polynucleotide comprises at least one insertion site for at least one transgene for delivery of gene therapy.

109. The method of claim 101, wherein the vector comprising the compstatin analog polynucleotide is selected from retrovirus, adenovirus, adeno-associated virus (AAV), and herpes simplex virus-1.

110. The method of claim 109, wherein the vector comprising the compstatin analog polynucleotide is an adeno-associated viral (AAV) vector.

111. The method of claim 110, wherein the AAV vector is an AAV1, AAV2, AAV3, AAV4, AAV5, AAV6, AAV7, AAV8, AAV9, AAV10, or AAV11 vector, or any variant or combination thereof.

112. The method of claim 101, wherein the vector comprising the compstatin analog polynucleotide comprises at least one other transgene for gene therapy.

113. The method of claim 101, wherein the vector comprising the compstatin analog polynucleotide is different from another vector comprising the other transgene for gene therapy, wherein the vectors are administered together.

114. The method of claim 101, wherein the vector comprising the compstatin analog polynucleotide is different from another vector comprising the other transgene for gene therapy, wherein the vectors are administered separately.

115. The method of any one of claims 101-114, comprising replacing the compstatin analog polynucleotide or the vector comprising the compstatin analog polynucleotide with the compstatin analog peptide itself.


SEQUENCE LISTING (18 sequences)

SEQ ID NO: 1 - protein, 13 aa, synthetic construct
  SITE 2..12: note = disulfide bond
ICVVQDWGHHRCT

SEQ ID NO: 2 - protein, 13 aa, synthetic construct
  MOD_RES 1: note = acetylation
  SITE 4: note = 1-methylated
  SITE 3..13: note = disulfide bond
ICVWQDWGAHRCT

SEQ ID NO: 3 - protein, 14 aa, synthetic construct
  MOD_RES 1: note = D-Tyrosine
  SITE 5: note = 1-methylated
  SITE 9: note = n-methylated
  SITE 14: note = methylated
  SITE 3..13: note = disulfide bond
YICVWQDWGAHRCI

SEQ ID NO: 4 - protein, 14 aa, synthetic construct
  VAR_SEQ 1: note = X is absent or is Tyr-Ile
  VARIANT 9: note = X is Ala or Glu
  VAR_SEQ 13: note = X is absent or is Ile
  VARIANT 14: note = X is Lys, Lys-Lys, or Lys-Lys-Lys
  SITE 2..12: note = disulfide bond
XCIWQDWGXHRCXX

SEQ ID NO: 5 - protein, 15 aa, synthetic construct
  SITE 3..13: note = disulfide bond
  SITE 10: note = X is Ala or Glu
  SITE 15: note = X is Lys, Lys-Lys, or Lys-Lys-Lys
YICIWQDWGXHRCIX

SEQ ID NO: 6 - protein, 17 aa, synthetic construct
  SITE 3..13: note = disulfide bond
YICIWQDWGAHRCIKKK

SEQ ID NO: 7 - protein, 17 aa, synthetic construct
  SITE 3..13: note = disulfide bond
YICIWQDWGEHRCIKKK

SEQ ID NO: 8 - protein, 16 aa, synthetic construct
  SITE 3..13: note = disulfide bond
  VARIANT 10: note = X is Ala or Glu
YICIWQDWGXHRCIKK

SEQ ID NO: 9 - protein, 15 aa, synthetic construct
  SITE 3..13: note = disulfide bond
  VARIANT 10: note = X is Ala or Glu
YICIWQDWGXHRCIK

SEQ ID NO: 10 - protein, 14 aa, synthetic construct
  SITE 3..13: note = disulfide bond
  VARIANT 10: note = X is Ala or Glu
YICIWQDWGXHRCI

SEQ ID NO: 11 - protein, 13 aa, synthetic construct
  SITE 3..13: note = disulfide bond
  VARIANT 10: note = X is Ala or Glu
YICIWQDWGXHRC

SEQ ID NO: 12 - protein, 15 aa, synthetic construct
  SITE 1..11: note = disulfide bond
  VARIANT 8: note = X is Ala or Glu
CIWQDWGXHRCIKKK

SEQ ID NO: 13 - protein, 14 aa, synthetic construct
  SITE 1..11: note = disulfide bond
  VARIANT 8: note = X is Ala or Glu
CIWQDWGXHRCIKK

SEQ ID NO: 14 - protein, 13 aa, synthetic construct
  SITE 1..11: note = disulfide bond
  VARIANT 8: note = X is Ala or Glu
CIWQDWGXHRCIK

SEQ ID NO: 15 - protein, 12 aa, synthetic construct
  SITE 1..11: note = disulfide bond
  VARIANT 8: note = X is Ala or Glu
CIWQDWGXHRCI

SEQ ID NO: 16 - protein, 11 aa, synthetic construct
  SITE 1..11: note = disulfide bond
  VARIANT 8: note = X is Ala or Glu
CIWQDWGXHRC

SEQ ID NO: 17 - DNA, 51 bp, synthetic construct
  source 1..51: mol_type = other DNA
  variation 30: note = N is G, A, T, or C
  variation 36: note = N is G, A, T, or C
  variation 3: note = Y is T or C
  variation 9: note = Y is T or C
  variation 21: note = Y is T or C
  variation 33: note = Y is T or C
  variation 39: note = Y is T or C
  variation 6: note = H is A, T, or C
  variation 12: note = H is A, T, or C
  variation 42: note = H is A, T, or C
  variation 18: note = R is G or A
  variation 45: note = R is G or A
  variation 48: note = R is G or A
  variation 51: note = R is G or A
  variation 27: note = N is G, C, A, or T
tayathtgyathtggcargaytggggngcncaycgntgyathaaraaraar

SEQ ID NO: 18 - DNA, 51 bp, synthetic construct
  source 1..51: mol_type = other DNA
  variation 6: note = H is A, T, or C
  variation 9: note = Y is T or C
  variation 12: note = H is A, T, or C
  variation 18: note = R is G or A
  variation 21: note = Y is T or C
  variation 27: note = N is G, A, T, or C
  variation 30: note = R is G or A
  variation 33: note = Y is T or C
  variation 36: note = N is G, A, T, or C
  variation 39: note = Y is T or C
  variation 42: note = H is A, T, or C
  variation 45: note = R is G or A
  variation 48: note = R is G or A
  variation 51: note = R is G or A
  variation 3: note = Y is T or C
tayathtgyathtggcargaytggggngarcaycgntgyathaaraaraar